(19) United States
(12) Patent Application Publication
(10) Pub. No.: US 2024/0420847 A1
(43) Pub. Date: Dec. 19, 2024

(54) SYSTEM AND METHODS FOR PERFORMING SALIVA-BASED DIAGNOSTIC SCREENINGS

(71) Applicant: CellectGen, Inc., Pasadena, CA (US)

(72) Inventors: Robert Gellibolian, Glendale, CA (US); Adam Markaryan, Glendale, CA (US)

(73) Assignee: CellectGen, Inc., Pasadena, CA (US)

(21) Appl. No.: 18/719,810

(22) PCT Filed: Jan. 2, 2023
(86) PCT No.: PCT/US2023/010013

§ 371 Date: Jun. 13, 2024

Related U.S. Application Data
(60) Provisional application No. 63/297,595, filed on Jan. 7, 2022.

(51) Int. Cl. G16H 50/30; G01N 33/68; G16H 10/60; G16H 50/70

(52) U.S. Cl. CPC G16H 50/30; G01N 33/6893; G16H 10/60; G16H 50/70; G01N 2333/96494

(57) ABSTRACT

A saliva-based diagnostic screening system and associated methods are disclosed for screening a volume of saliva of a patient for a presence or absence of active periodontal disease.

### RELATED APPLICATIONS

This application claims priority and is entitled to the filing date of U.S. provisional application Ser. No. 63/297,595, filed on Jan. 7, 2022. The contents of the aforementioned application are incorporated herein by reference.

### BACKGROUND

The subject of this patent application relates generally to medical diagnostics, and more particularly to a system and associated methods for performing saliva-based diagnostic screenings.

Applicant hereby incorporates herein by reference any and all patents and published patent applications cited or referred to in this application.

By way of background, in 2017, the American Academy of Periodontology (“AAP”) revised the 1999 periodontal classification system to be consistent with the current knowledge on pathophysiology, improving diagnosis and treatment. It provides a framework for case definition based on latest consensus evidence and can be adapted as new evidence emerges. It suggests a case definition system that can be implemented in real world clinical practice, research, and population level surveillance. The method of making a diagnosis in oncology served as a model for the new periodontal classification system whereby the diagnosis is based on two parameters: 1) staging, which captures the severity of the disease process, the extent and distribution of the pathologic damage, the complexity of management; and 2) grading, which relates to the rate of disease progression and risk factors that facilitate disease progression. Incorporating this medical model into the diagnosis of periodontitis allows for a more individualized diagnosis for each case. It considers the severity, extent, and distribution of the destruction of periodontal tissues and the rate of progression of disease and responsiveness to therapy.

One of the key aspects of this classification is how to assess and account for variability in the rate of periodontal disease progression. To date, clinical measurements (and known risk factors) are the only tools used to predict progression and are included in the new periodontal disease classification system. While this represents a positive step in the right direction, the system's continued and exclusive reliance on an array of subjective clinical measures will result in a high level of heterogeneity in the perceived accuracy of case definitions, thus falling short of the promise of moving periodontal diagnostics into the era of “P4 medicine”—participatory, predictive, preventive, and personalized. Moreover, this view limits and focuses treating disease when it is clinically identifiable and visible and fails to provide any meaningful information on the current status of disease activity or identify patients and sites at risk of disease progression.

Disease progression entails measurement of two inherent parameters: 1) disease activity levels, which is a measure of the magnitude of periodontal tissue destruction; and 2) the element of time, which measures the change in this activity over a specific period. But predicting (and measuring) disease progression accurately requires an appreciation of one of the most central tenets in health and disease: biochemical/molecular changes always precede the onset of clinical signs and symptoms. So, while clinical measures are important, they reflect biological events (e.g., tissue destruction) that have already happened and are subject to observer bias. As such, they have limited prognostic value for predicting disease progression in biologically relevant timelines (days or months), when interventions can have the most impact. Moreover, any treatment planning based on this information would be reactive at best.

Adding biomarker information to clinical datasets can enhance the mechanistic understanding of intra-patient differences in disease trajectory and reveal differences in important clinical outcomes, facilitating transition into precision periodontics.

Periodontal disease is one of the leading causes of tooth loss in adults and affects approximately half of the U.S. adult population. Periodontal disease begins with gingivitis, the localized inflammation of the gingiva. If this early stage is left untreated, the gums can morph into advanced periodontal disease, where infection, tooth loss, and serious systemic health problems are very real threats. The early stages of periodontal disease are often symptomless and “silent,” and a significant number of affected patients are unaware and do not seek professional care until they exhibit symptoms and risk losing one or multiple teeth, affecting their nutrition, quality of life and self-esteem as well as imposing huge socio-economic impacts and healthcare costs. A recent Global Burden of Disease Study indicates that severe periodontitis is the 6th most prevalent disease worldwide, with an overall prevalence of 11.2% and around 743 million people affected; and the global burden of periodontal disease increased by 57.3% from 1990 to 2010.

Periodontal tissues are mostly made up of type I collagen. The proteolytic enzyme believed to be responsible for the active periodontal soft and hard tissue degeneration is matrix metalloproteinase (“MMP-8”), also known as collagenase-2 or neutrophil collagenase. MMP-8 is a member of the collagenase protease group. Structurally related but genetically distinct MMPs are Ca²⁺- and Zn²⁺-dependent endopeptidases capable of degradation of almost all extracellular matrix and basement membrane protein components both in physiologic repair and pathologic destruction of tissues, such as a breakdown of extracellular matrix in embryonic development, wound healing, and tissue remodeling. MMP-8 is an important mediator of tissue destruction in inflammatory diseases such as periodontal disease. Biologically, MMP-8 is generally found in two main forms: a latent (inactive) form of the enzyme (“latent MMP-8”), and an active form of the enzyme (“active MMP-8”). The relationship between these two forms can be described by the following equation:

$$total MMP - 8 = ( active MMP - 8 ) + ( latent MMP - 8 )$$

Thus, the total MMP-8 is comprised of both the latent MMP-8 and the active MMP-8. The latent MMP-8 (75 kDa) contains a pro-domain that masks accessibility of the active site. Studies of anaerobic periodontal infections have shown that active MMP-8 in gingival crevicular fluid is associated with the degradation of periodontal tissues in progressive periodontitis whereas the latent enzyme is predominant in gingivitis. MMP-8 is produced by various tissues and cells and is secreted by neutrophils. It is synthesized as pre-proMMP-8, from which the signal peptide is removed during translation to generate proMMP-8, which is then secreted in its latent (inactive) form. Once latent MMP-8 is secreted (or degranulated) from neutrophils as a 75 kDa molecule in pro-form (latent MMP-8), it is reduced to its 57 kDa active form (active MMP-8), which is the form believed to be involved in destruction of soft (gums) and hard (bone) tissues and leads to bone loss. Although the mechanism of activation of MMPs in vivo is not completely understood, in vitro studies have demonstrated that collagenases can be activated by a diverse range of agents that remove or modify the conformation of the pro-domain. Removal of the pro-domain by enzymes of host or bacterial origin results in a reduction of the molecular mass of the MMPs.

Different proteolytic enzymes cleave at different sites in the proenzyme domain, generating different sizes of active enzyme with different levels of enzyme activity. Notably, the correct folding of the activated MMP-8 is critical for enzyme activity and stability. Full activation occurs only when the correct amino-terminal amino acid is generated following proteolytic cleavage by intermolecular or complex autocatalytic reactions.

In periodontitis, levels of MMP-8 increase in the gingival crevicular fluid (“GCF”) exudate and consequently in samples of whole saliva. Moreover, active MMP-8, but not the latent enzyme, is associated with periods of active connective tissue destruction and a clinical diagnosis of periodontitis. As such, saliva becomes an important, non-invasive, and accessible fluid from which MMP-8 (and other markers) can be measured.

There are three main challenges in treating periodontitis. The first challenge in treating periodontal disease is a timely and accurate diagnosis. Disease diagnosis and treatment in its earliest stages will prevent future breakdown. Because the disease is painless, patients rarely seek care. Thus, it is not uncommon for the disease to go undiagnosed until progression has reached moderate to advanced degrees of severity, characterized by obvious radiographic bone loss and/or tooth mobility. The second challenge is diagnosing and properly controlling the local environment and the host response, which contribute to the variabilities associated with disease progression and response to treatment. Successful management and prevention of periodontitis can be very challenging. Thus, it is imperative that medical service providers are aware of the patient's susceptibility to disease, progression of disease, and potential response to therapy before initiating a treatment plan. The third challenge in the treatment of periodontitis involves long-term maintenance. This phase of therapy is known as supportive periodontal therapy, or periodontal maintenance (“PM”). The challenge during this phase of therapy is focused on maintaining patient motivation and compliance, management of all risk factors, and finally making the appropriate decisions regarding re-treatment, when indicated.

At the present time, the diagnosis and classification of periodontal disease are almost entirely based on clinical assessments. Quantitative and qualitative assessments of salivary markers of disease, and/or gingival crevicular fluid and subgingival microflora has been shown to provide very useful information about the patient's periodontal disease status and progression of disease. These supplemental risk-assessment tests are particularly valuable in establishing the endpoint of therapy prior to placing patients on a periodontal maintenance program. But despite this need, the field still relies exclusively on clinical measures of disease, mainly because it has been a challenge integrating supplemental molecular data into the workflow.

Currently, diagnosis of chronic gum disease requires recording multiple clinical parameters such as bleeding on probing (“BOP”), probing pocket depth (“PPD”), and clinical attachment loss (“CAL”) at six locations per tooth (whether affected or not). This results in a laborious diagnostic process that is also dependent on the expertise of the clinician, making the process very subjective. Furthermore, this process needs to be regularly repeated at recall visits to monitor the disease course. Radiographic (or X-ray) is an important adjunct to periodontal probing to differentiate between true disease and gingivitis. However, X-ray reveals changes only after 30% to 50% of the bone loss has already occurred, routinely leading to underestimation in the levels of bone loss. The limitations of clinical measures are two-fold: they are indicators of past periodontal disease rather than present (or predictors of future) disease activity; and due to their low sensitivity and low positive predictive value, measurement of clinical parameters alone are inadequate for predicting susceptible individuals who might be at risk of periodontitis in the future. The best clinical predictor of gingival inflammation to date is BOP; but false positives associated with this method make exclusive reliance on this clinical parameter inaccurate.

As an alternative to clinical diagnosis techniques, there are several molecular tests on the market. One such molecular test measures harmful oral bacteria in order to determine a person's genetic susceptibility to gum disease. However, such tests tend to not be user-friendly or cost-effective, while also being time-consuming. Another such molecular test is a lateral flow test for detection of the active MMP-8 concentration in saliva. However, it requires an instrument to quantify results, which means it cannot be used by a consumer at home. It also only detects one form of biomarker, active MMP-8. The validity of such tests is also questionable for two reasons. First, the detection of antibodies is targeted towards the middle part activation products or fragments (20 to 35 kDa) of MMP-8. Once MMP-8 is secreted (or degranulated) from neutrophils as a 75 kDa molecule in pro-form (latent MMP-8), it is reduced to its 57 kDa active form (active MMP-8). The 20-27 kDa fragments of MMP-8 are degradation end-products of this 57 kDa active MMP-8 activation and are without collagenase activity. As such, the fragments detected by such tests cannot take part in active collagen-1 breakdown, which is believed to be responsible for soft and hard tissue breakdown (i.e., bone loss). This fact brings into question the validity of measuring levels of these fragments in saliva samples of patients. Secondly, detection of each of the 20 to 35 kDa fragment would lead to an overestimation of levels as each fragment would register a separate signal in the test, which would be additive in the result. This is compared to one signal that one would expect for each 57 kDa active MMP-8 molecule. This would limit the ability to compare levels of different forms of the MMP-8 in each patient sample or perform ratiometric analysis. Furthermore, such an indirect measurement suffers from a major limitation: the data cannot be used to make comparative extrapolations with other markers, such as total MMP-8, and/or test plausible biological hypotheses.

The current lack of appropriate molecular and data analytics tools presents some major challenges to proper integration within current clinical diagnostic workflow. For example, without the availability of high-quality and specific antibodies to both the active (or latent) and total forms of MMP-8, the prognostic value of MMP-8 testing remains very limited. Currently, most testing and validations have been done with total MMP-8 only or activation byproducts of MMP-8 only. Additionally, whole saliva levels of MMP-8 offer a broad view of a patient's oral health status and offers limited, if any prognostic clinical value at each clinical site/tooth. Given that dentists and hygienists collect around six measurable clinical data points—per clinical parameter such as probing pocket depth (PPD), bleeding on probing (BOP), etc.—per tooth, and make decisions based on these site-specific clinical data, it's challenging to integrate whole mouth MMP-8 data into the workflow to inform and guide dentists on next steps or perform proper risk assessment and patient stratification. With six sites per tooth, dentists would need to collect approximately 192 samples per patient per visit in order to obtain whole mouth MMP-8 data, which is not only impractical, but also time and cost prohibitive. There is also variability in number of cells shed in saliva. These cells are the neutrophils (and other) which are present in GCF and express and store the pre-latent form of the MMP-8 in granulocytes that are then released in latent form when needed. As such these tests do not standardize how much secreted versus cell-based MMP-8 one can detect in whole saliva, especially between sample collections and if cell numbers vary. This may be why total MMP-8 levels varies so widely among patients.

Accordingly, there remains a need for a system and associated methods that can predict site (or tooth) specific levels of active and total MMP-8 using a single saliva sample collection, which would save time, money and provide the solution needed for a proper clinical diagnostic workflow and workup. Aspects of the present invention fulfill these needs and provide further related advantages as described in the following summary.

It should be noted that the above background description includes information that may be useful in understanding aspects of the present invention. It is not an admission that any of the information provided herein is prior art or relevant to the presently claimed invention, or that any publication specifically or implicitly referenced is prior art.

### SUMMARY

Aspects of the present invention teach certain benefits in construction and use which give rise to the exemplary advantages described below.

The present invention solves the problems described above by providing a saliva-based diagnostic screening system and associated methods for screening a volume of saliva of a patient for a presence or absence of active periodontal disease. In at least one embodiment, for each of an at least one patient visit to a medical service provider of the patient, a volume of the patient's saliva is collected; a quantity of total MMP-8 (“tMMP-8”) along with a quantity of at least one of active MMP-8 (“aMMP-8”) and latent MMP-8 (“IMMP-8”) present in the collected saliva is determined; a clinical measurement table is populated containing a plurality of probing pocket depth (“PPD”) values and corresponding bleeding on probing (“BOP”) values for an at least one site within a mouth of the patient as measured by a medical service provider for the patient; a clinical distribution table is populated containing a distribution of a quantity of BOP sites relative to each PPD value in the clinical measurement table; a weighted average PPD is calculated for each site where bleeding on probing is detected using the formula

$$f ( x ) = {\sum }_{x = 1}^{14} ( PP D ( x ) \times BOP Sites ( x ) ) / {\sum }_{x = 1}^{14} BOP Sites ( x )$$

where x is the PPD value corresponding to each site; a weighted average PPD is calculated for each site where bleeding on probing is not detected using the formula

$$f ( x ) = {\sum }_{x = 1}^{14} ( PP D ( x ) \times noBOP Sites ( x ) ) / {\sum }_{x = 1}^{14} noBOP Sites ( x )$$

where x is the PPD value corresponding to each site; a distribution of total MMP-8 quantities is calculated for each site where bleeding on probing is detected using the formula

$$f ( x ) = ( \frac{BOP Sites ( x )}{total sites} ) \times ( tMMP - 8 concentration )$$

where x is the PPD value corresponding to each site; a distribution of total MMP-8 quantities is calculated for each site where bleeding on probing is not detected using the formula

$$f ( x ) = ( \frac{noBOP Sites ( x )}{total sites} ) \times ( tMMP - 8 concentration )$$

where x is the PPD value corresponding to each site; a total MMP-8 level table is populated containing the calculated weighted average PPD and distribution of total MMP-8 quantities for each site; an MMP-8 distribution percentage table is populated containing a distribution percentage of active MMP-8 and a distribution percentage of latent MMP-8 for each site; an oral inflammatory burden (“OIB”) is calculated based on the level of total MMP-8 in the collected saliva; a disease activity (“DA”) is calculated, representing a relative level of active MMP-8 compared to the level of total MMP-8 in the collected saliva, using the formula

$$D A = ( \frac{a MMP - 8}{t MMP - 8} )$$

where aMMP-8 is the amount of active MMP-8 concentration in the collected saliva and tMMP-8 is the amount of total MMP-8 concentration in the collected saliva; a rate of change is calculated for each of the PPD, OIB and DA values between the current patient visit and a previous patient visit; and at least one of a qualitative risk stratification table and a quantitative risk stratification table is populated based on the calculated rate of change for each of the PPD, OIB and DA values between the current patient visit and a previous patient visit. Accordingly, one or both of the qualitative risk stratification table and quantitative risk stratification table can be used by the medical service provider to accurately and objectively predict the patient's future risk of periodontal disease.

Other features and advantages of aspects of the present invention will become apparent from the following more detailed description, taken in conjunction with the accompanying drawings, which illustrate, by way of example, the principles of aspects of the invention.

### BRIEF DESCRIPTION OF THE DRAWINGS

The accompanying drawings illustrate aspects of the present invention. In such drawings:

FIG. 1 is a simplified schematic view of an exemplary saliva-based diagnostic screening system, in accordance with at least one embodiment;

FIGS. 2 and 3 are flow diagrams of an exemplary method for performing a saliva-based diagnostic screening, in accordance with at least one embodiment;

FIG. 4 is an illustration of an exemplary clinical measurement table, in accordance with at least one embodiment;

FIG. 5 is an illustration of an exemplary clinical distribution table, in accordance with at least one embodiment;

FIG. 6 is an illustration of an exemplary total MMP-8 level table, in accordance with at least one embodiment;

FIG. 7A is an illustration of an exemplary MMP-8 distribution percentage table, in accordance with at least one embodiment;

FIG. 7B is a graph depicting the data contained in the MMP-8 distribution percentage table of FIG. 8A, in accordance with at least one embodiment;

FIG. 8 is an illustration of an exemplary MMP-8 distribution amount table, in accordance with at least one embodiment;

FIG. 9 is an illustration of an exemplary qualitative risk stratification table, in accordance with at least one embodiment;

FIG. 10 is an illustration of an exemplary quantitative risk stratification table, in accordance with at least one embodiment;

FIG. 11 is an illustration of an exemplary diagnostic table, in accordance with at least one embodiment;

FIG. 12 is an illustration of an exemplary quantitative risk score table, in accordance with at least one embodiment; and

FIG. 13 is an illustration of an exemplary table containing minimum rate of change, maximum rate of change and distribution difference values for each of a probing pocket depth, an oral inflammatory burden, and a disease activity across a plurality of patients, in accordance with at least one embodiment.

The above-described drawing figures illustrate aspects of the invention in at least one of its exemplary embodiments, which are further defined in detail in the following description. Features, elements, and aspects of the invention that are referenced by the same numerals in different figures represent the same, equivalent, or similar features, elements, or aspects, in accordance with one or more embodiments.

### DETAILED DESCRIPTION

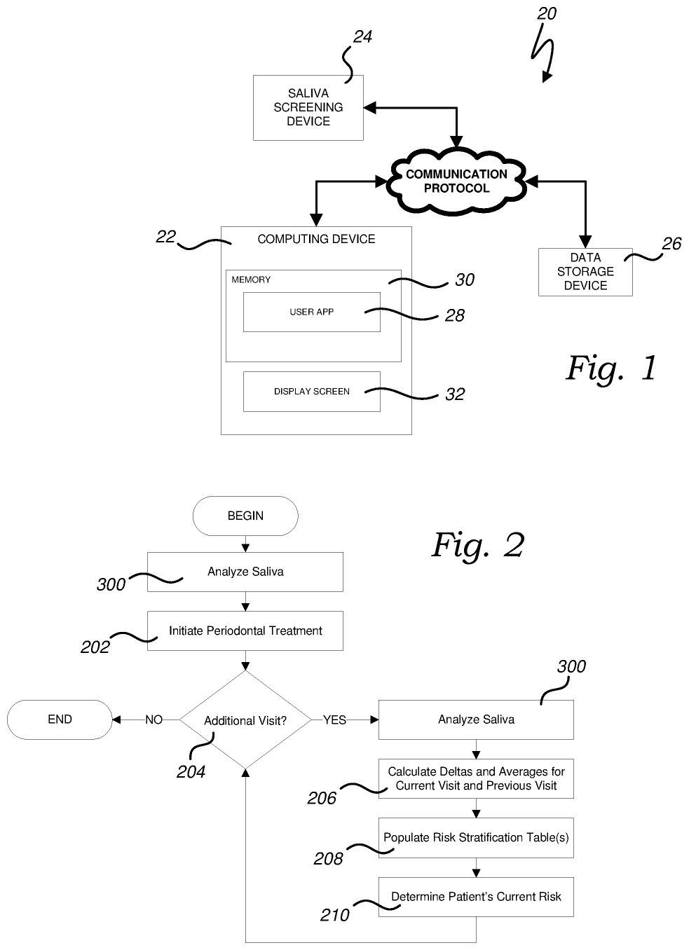

Turning now to FIG. 1, there is shown a simplified schematic view of an exemplary saliva-based diagnostic screening system 20 configured for screening a volume of saliva of a patient for the presence or absence of active periodontal disease using patient level clinical and biomarker data, as discussed further below. In at least one embodiment, as also discussed further below, the system 20 utilizes predictive modeling methods to derive site-specific (or tooth-specific) information from single saliva data.

Accordingly, in at least one embodiment, the system 20 provides an at least one computing device 22 configured for receiving and processing select data obtained by an at least one saliva screening device 24 from the patient's saliva. In that regard, it should be noted that while certain types of saliva screening devices 24 may be discussed herein for illustrative purposes (such as the biofluid-based diagnostic screening system described in U.S. 63/297,595 and PCT/US2023/010012, for example, the details of which are incorporated herein by reference), in further embodiments, any other types of devices, sensors, techniques or combinations thereof, now known or later developed, capable of obtaining the data necessary from the patient's saliva for subsequent processing by the computing device 22 may be substituted. In at least one embodiment, the computing device 22 and the at least one saliva screening device 24 are one and the same—as such, it is intended that those terms as used herein are to be interchangeable with one another. In at least one alternate embodiment, the saliva screening device 24 is omitted, such that the patient's saliva data is provided to the computing device 22 through other means. In at least one embodiment, the system 20 further provides an at least one data storage device 26 in selective communication with the computing device 22 and configured for storing said data obtained by the at least one saliva screening device 24 and processed by the computing device 22, along with certain other data as discussed further below. In at least one embodiment, the computing device 22 and data storage device 26 are also one and the same—as such, it is intended that those terms as used herein are to be interchangeable with one another as well.

At the outset, it should be noted that communication between each of the computing device 22, saliva screening device 24, and data storage device 26 may be achieved using any wired- or wireless-based communication protocol (or combination of protocols) now known or later developed. As such, the present invention should not be read as being limited to any one particular type of communication protocol, even though certain exemplary protocols may be mentioned herein for illustrative purposes. It should also be noted that the term “computing device” is intended to include any type of computing or electronic device, now known or later developed, capable of substantially carrying out the functionality described herein—such as desktop computers, mobile phones, smartphones, laptop computers, tablet computers, personal data assistants, gaming devices, wearable devices, etc. As such, the system 20 should not be read as being limited to use with any one particular type of computing or electronic device, even though certain exemplary devices may be mentioned or shown herein for illustrative purposes.

In at least one embodiment, the computing device 22 contains the hardware and software necessary to carry out the exemplary methods for performing saliva-based diagnostic screenings, as described herein. Furthermore, in at least one embodiment, the computing device 22 comprises a plurality of computing devices selectively working in concert with one another to carry out the exemplary methods for performing saliva-based diagnostic screenings, as described herein. In at least one embodiment, the computing device 22 provides a user application 28 residing locally in memory 30 on the computing device 22, the user application 28 being configured for selectively communicating with the at least one saliva screening device 24 in at least one embodiment, as discussed further below. It should be noted that the term “memory” is intended to include any type of electronic storage medium (or combination of storage mediums) now known or later developed, such as local hard drives, RAM, flash memory, secure digital (“SD”) cards, external storage devices, network or cloud storage devices, integrated circuits, etc. In at least one embodiment, the computing device 22 provides an at least one display screen 32 configured for displaying select data, as discussed in detail below. In at least one such embodiment, the display screen 32 is a touchscreen.

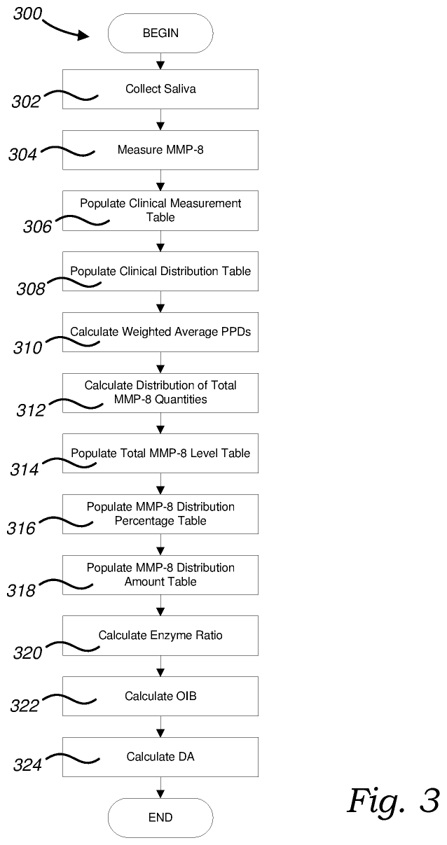

In use, in at least one embodiment, the system 20 is capable of screening a volume of the patient's saliva for the presence or absence of active periodontal disease using patient level clinical and biomarker data. In at least one embodiment, the system 20 screens the patient's saliva for both total MMP-8 and active MMP-8. However, it should be noted that in further embodiments, other biomarkers (now known or later discovered) may be used either in place of or in addition to MMP-8, such as MMP-1, MMP-2, MMP-9, and MMP-13, or even levels of various bacterial species (now known or later discovered), for example. In at least one embodiment, as illustrated in the flow diagram of FIGS. 2 and 3, during each visit of the medical service provider by the patient, a volume of the patient's saliva is collected using a saliva collection device (302). In at least one embodiment, the saliva collection device is a biofluid collection and filtration device similar to those described in U.S. Pat. Nos. 9,816,087, 10,302,535, and 10,830,677, the details of which are incorporated herein by reference. However, in further embodiments, the saliva collection device may be any other device, now known or later developed, capable of collecting a volume of saliva. In at least one embodiment, about 1 milliliter of the patient's saliva (either mouth wash or straight saliva) is collected by the saliva collection device; however, in further embodiments, any other volumes of the patient's saliva may be collected.

In at least one embodiment, the collected saliva is analyzed to determine a quantity of total MMP-8 along with a quantity of active MMP-8 and/or latent MMP-8 present in the collected saliva (304). Given that total MMP-8 is the sum of active MMP-8 and latent MMP-8, determining a quantity of at least two of those MMP-8 forms allows for the determination of the third of those MMP-8 forms. In at least one further embodiment, the collected saliva is further analyzed to determine a quantity of an at least one ubiquitous protein, such as actin or most abundant salivary protein alpha amylase, for example, which allows for the normalization of MMP-8 levels in the patient's saliva and, in turn, a more accurate analysis. In a bit more detail, in at least one such embodiment, a specific activity of a given MMP-8 form (depicted as milligrams of the MMP-8 form per milligram of ubiquitous protein) is calculated using the following formula:

$$Specific Activity = \frac{MMP - 8}{ubiquitous protein}$$

In at least one embodiment, the collected saliva is analyzed (by the saliva screening device 24 or other means) using an enzyme-linked immunosorbent assay (“ELISA”) test, through which the collected saliva is cleaned (by centrifugation and/or filtration) and two to three serial dilutions (1:0, 1:10, and 1:100, for example) of the collected saliva are then used to assay using ELISA. In at least one alternate embodiment, the collected saliva is analyzed (by the saliva screening device 24 or other means) using a lateral flow assay (“LFA”) test, through which the collected saliva is cleaned (by centrifugation and/or filtration), an appropriate volume (one to five drops, for example) of the cleaned saliva is deposited onto a test strip, an appropriate volume (a few drops, for example) of a buffer is subsequently deposited onto the test strip, and the results are analyzed with an appropriate reader (e.g., the saliva screening device 24, the user application 28 of the computing device 22, a colorimetric/fluorometric reader, a visual reader, etc.). In still further embodiments, any other devices, sensors, techniques or combinations thereof, now known or later developed, capable of determining the quantity of total MMP-8 and the quantity of active MMP-8 present in the collected saliva may be substituted. In at least one embodiment, during a first visit to the medical service provider, the patient's saliva is collected prior to performing any treatments or clinical measurements on the patient, as such activities could cause bleeding in the patient's gums and interfere with the saliva analysis.

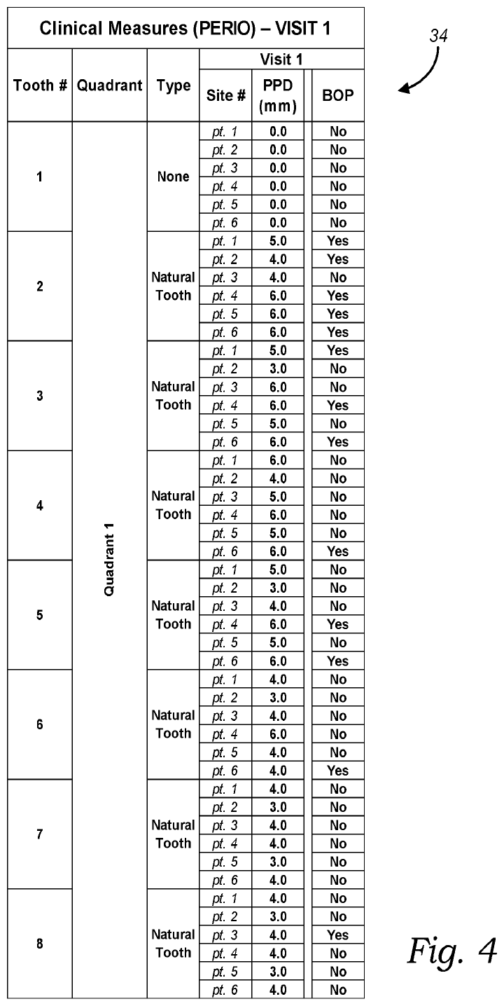

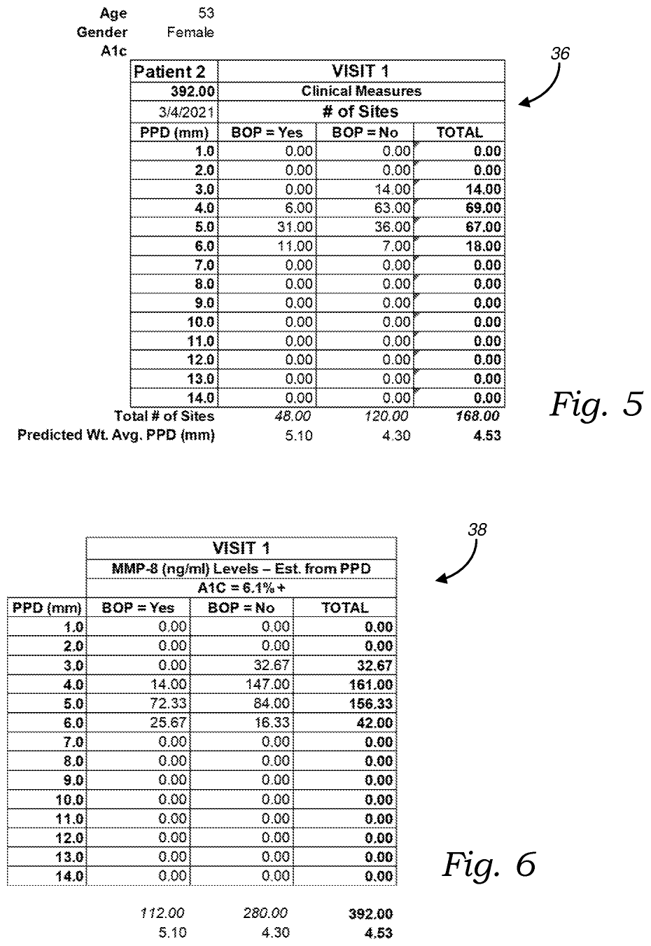

In at least one embodiment, after the patient's saliva has been collected, the user application 28 populates a clinical measurement table 34 that contains a plurality of probing pocket depth (“PPD”) values and corresponding bleeding on probing (“BOP”) values for at least one tooth of a given patient as measured by the medical service provider for said patient for each visit (306). However, it should be noted that in further embodiments, other clinical measures (now known or later discovered) may be used either in place of or in addition to PPD, such as clinical attachment loss (“CAL”) or bone loss, for example. It should also be noted that the term “medical service provider” is intended to generally include (but is in no way limited to) physicians, dentists, nurses, clinicians, hospitals, clinics and any other type of medical professional or medical entity who may be tasked or otherwise authorized to provide medical services to the at least one patient—even though certain types of medical professionals or entities (such as dentists and dental offices) may be expressly mentioned herein for illustrative purposes. Additionally, while the term “table” is used herein to describe certain exemplary data structures, in at least one embodiment, any other suitable data type or data structure, or combinations thereof, now known or later developed, capable of storing the appropriate data, may be substituted. Thus, the present invention should not be read as being so limited. An exemplary clinical measurement table 34 for eight of the patient's teeth during a first visit is illustrated in FIG. 4. In at least one embodiment, the user application 28 populates a clinical distribution table 36 that contains a distribution of a quantity of BOP sites (i.e., BOP=Yes and BOP=No) relative to each PPD value in the clinical measurement table 34 (308). An exemplary clinical distribution table 36 is illustrated in FIG. 5. For example, the patient represented in FIGS. 4 and 5 has a total of 67 sites with a PPD value of 5.0 mm, 31 of which bleed and 36 that do not. In total, the patient represented in FIGS. 4 and 5 has 168 sites, 48 of which bleed and 120 do not.

In at least one embodiment, based on the data contained in the clinical distribution table 36, the user application 28 calculates a weighted average PPD for all sites for the patient during that single visit (310) using the following formulas:

$$BOP = Yes : f ( x ) = {\sum }_{x = 1}^{14} ( P P D ( x ) \times BOP Sites ( x ) ) / {\sum }_{x = 1}^{14} BOP Sites ( x )$$

$$BOP = No : f ( x ) = {\sum }_{x = 1}^{14} ( PP D ( x ) \times noBOP Sites ( x ) ) \text{} / {\sum }_{x = 1}^{14} noBOP Sites ( x )$$

where x is the corresponding PPD value. Accordingly, in the example illustrated in FIGS. 4 and 5, the average PPD for sites with BOP (i.e., BOP=Yes) and with no BOP (i.e., BOP=No) is 5.10 millimeters and 4.30 millimeters, respectively, with an overall average PPD of 4.53 millimeters.

In at least one embodiment, the user application 28 calculates a distribution of total MMP-8 quantities per site for each PPD with or without BOP for the patient during that single visit (312) using the following formulas:

$$BOP = Yes : f ( x ) = ( \frac{BOP Sites ( x )}{total sites} ) \times ( tMMP - 8 concentration )$$

$$BOP = No : f ( x ) = ( \frac{noBOP Sites ( x )}{total sites} ) \times ( tMMP - 8 concentration )$$

where x is the corresponding PPD value. In at least one embodiment, the resulting calculations are then used by the user application 28 to populate a total MMP-8 level table 38 (314), as illustrated in FIG. 6. Accordingly, in the example illustrated in FIGS. 4-6, the estimated total MMP-8 levels for sites with BOP and with no BOP is 112.0 ng/ml and 280.0 ng/ml, respectively. Additionally, using the MMP-8 distribution, the average PPD for sites (using MMP-8 distribution data) with BOP and with no BOP is calculated by the user application 28 to be 5.10 millimeters and 4.30 millimeters, respectively, with an overall average PPD of 4.53 millimeters. This is not surprising given that site-specific MMP-8 distribution is calculated using the data from the clinical measurement table 34 and clinical distribution table 36.

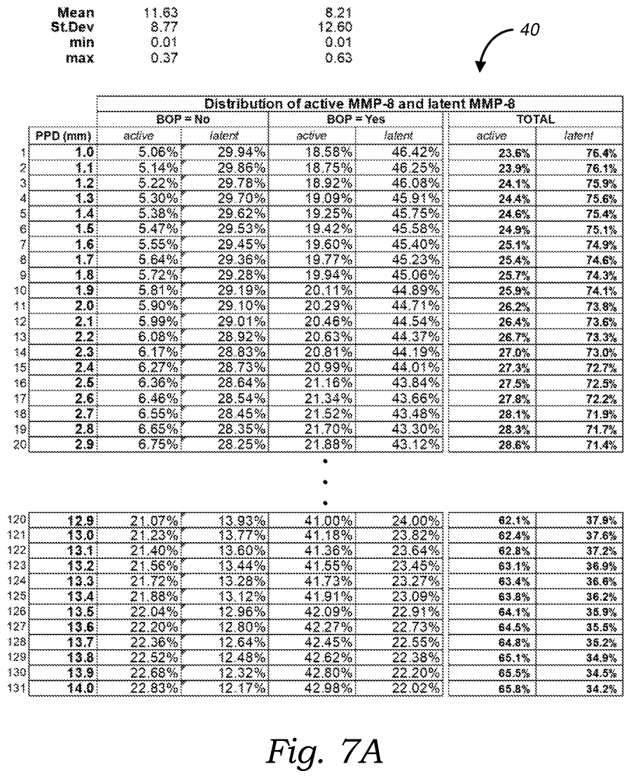

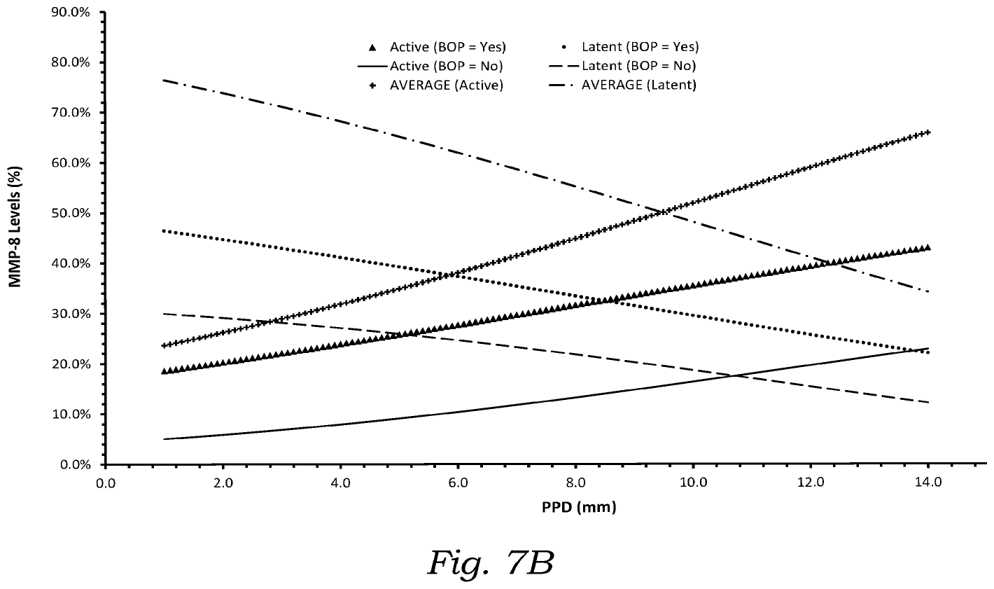

In at least one embodiment, given that the active MMP-8 in gingival crevicular fluid is associated with the degradation of periodontal tissues in active periodontitis and that such destruction is normally associated with bleeding at the site of damage, the user application 28 concludes that a percentage of the total MMP-8 in sites that bleed (BOP=Yes) is comprised of active MMP-8 and the remaining portion is latent MMP-8. In sites that do not bleed (BOP=No), this percentage is different. Thus, this relationship is not binary from 0 to 1 with BOP constituting 100% active MMP-8 and no BOP constituting 0%. In other words, assuming that approximately 100% of the MMP-8 in bleeding sites (BOP=Yes) is active MMP-8 and approximately 100% of the MMP-8 in non-bleeding sites (BOP=No) is latent MMP-8 is wrong, as it would suggest that a bleeding site with a PPD value of 4 millimeters has the same amount of active enzyme as a bleeding site with a PPD value of 8 millimeters, which would be wrong. In at least one embodiment, a more accurate way is for the user application 28 to estimate the levels of active MMP-8 and latent MMP-8 in both bleeding (BOP=Yes) and non-bleeding (BOP=No) sites for all PPD values. Accordingly, in at least one embodiment, before assessing ratiometric measures, the user application 28 first determines this distribution. In the example above, it would be erroneous to conclude that all the MMP-8 for sites that bleed (112 ng/ml) are comprised of active MMP-8. Similarly, it would be erroneous to conclude that all the MMP-8 for sites that do not bleed (280 ng/ml) are comprised of latent MMP-8. In at least one embodiment, estimation of how much active MMP-8 or latent MMP-8 there is in sites that bleed vs. sites that do not bleed depends on the clinical measure of pocket depth (PPD); a site having a PPD value of 2.0 millimeters with BOP has less active MMP-8 and less risk of tissue destruction than a site having a PPD value of 8.0 millimeters with BOP. In at least one embodiment, this relationship is mathematically governed based on a normal regression curve (S-Curve) using the following formula:

$$f ( x ) = \frac{{L}_{Max}}{( 1 + {e}^{- k ( x - {x}_{0} )} )}$$

where e is the natural logarithm base, x₀ is the x-value of the curve's midpoint, L_{Max} is the curve's maximum value, and k is the slope of the curve. However, in further embodiments, the distribution percentages of active MMP-8 and latent MMP-8 for each site may be calculated using any other appropriate mathematical formula, now known or later developed. In at least one embodiment, the resulting calculations are then used by the user application 28 to populate an MMP-8 distribution percentage table 40 (316), as illustrated in FIG. 7A. The data contained in the MMP-8 distribution percentage 40 table is also depicted in the graph of FIG. 7B for illustrative purposes. Accordingly, in the example illustrated in FIGS. 7A and 7B, for a PPD value of 1.0 millimeter in sites that do not bleed (BOP=No), the model generated by the user application 28 predicts that 5.06% of the MMP-8 burden is comprised of active MMP-8 and 31.94% is latent MMP-8. In contrast, for the same PPD value in sites that do bleed (BOP=Yes), the model generated by the user application 28 predicts that 18.58% of the burden is comprised of active MMP-8 and 44.42% is latent MMP-8. Based on best fit, the model generated by the user application 28 also shows that sites that do not bleed (BOP=No) contribute less to the total MMP-8 burden than sites that do bleed (BOP=Yes). This is as expected since a site with a specific PPD value that bleeds is expected to have more active MMP-8 than a site with the same PPD value that does not bleed (in this example, 18.58% versus 5.06%). This trend is expected to follow a normal distribution with higher PPD values.

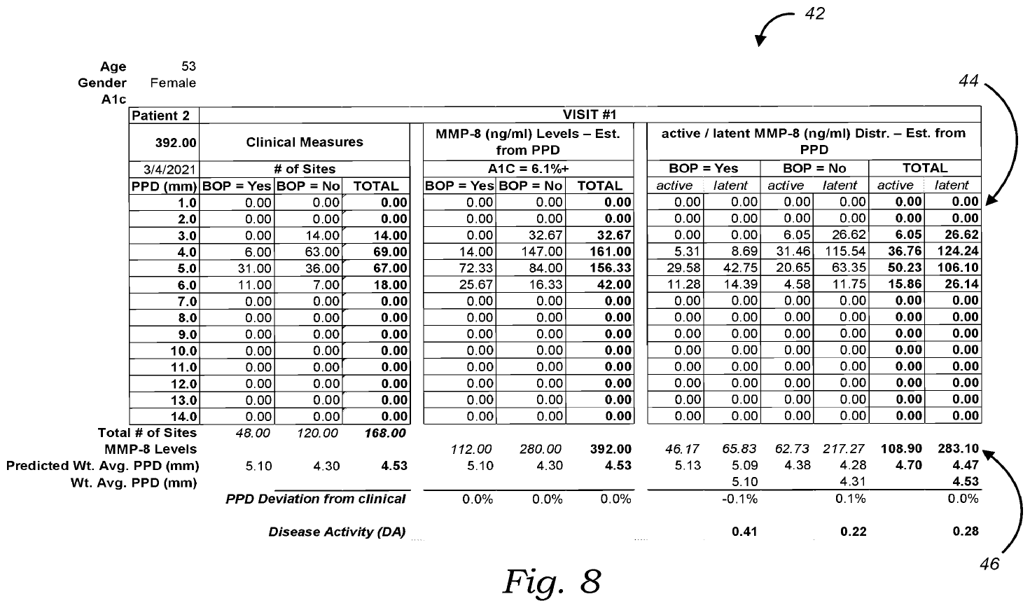

In at least one embodiment, the user application 28 populates an MMP-8 distribution amount table 42 (318) using the data contained in the clinical distribution table 36, total MMP-8 level table 38 and MMP-8 distribution percentage table 40, as illustrated in FIG. 8. In at least one embodiment, the MMP-8 distribution amount table 42 contains two levels of data: a whole mouth level 44 of data, and a site-specific level 46 of data. The whole mouth level 44 of data is the one-degree layer data from a whole mouth perspective, which provides the medical service provider with a high-level view of how the patient is responding to a specific therapy, or how well a particular therapy worked in lowering the burden of disease (see ratiometric measures discussed below below). In at least one embodiment, the user application 28 empirically measures and tests levels of the active MMP-8 and total MMP-8 in the patient's saliva and utilizes select ratiometric measures (as discussed below) to determine overall periodontal health. However, it is important to note that relying exclusively on this one-degree layer data can be misleading as it only represents a median/average of the whole mouth, and with 160 to 192 sites (representing 32 teeth), it may underestimate and mask one or a few problematic sites (having high PPD values and/or BOP) that are not weighed adequately in the average calculation. Thus, in at least one embodiment, the user application 28 further calculates and utilizes site-specific level 46 data as a further determining factor in challenging periodontal cases. The site-specific level 46 of data is the two-degree layer data, which provides a more granular view of what is happening at each site. The site-specific level 46 of data complements the whole mouth level 44 of data. However, the site-specific level 46 of data does not lend itself to be easily measured empirically and as such is best done using a predictive algorithm such as the one utilized by the user application 28. Accordingly, in the example illustrated in FIGS. 4-8 and the corresponding model generated by the user application 28, 112.0 ng/ml (out of the 392.0 ng/ml) of the total MMP-8 is in sites that bleed (BOP=Yes) and 280.0 ng/ml in sites that do not bleed (BOP=No). Additionally, in sites that bleed (BOP=Yes), a total of 46.17 ng/ml (out of the 112.0 ng/ml) is active MMP-8 and 65.83 ng/ml is latent MMP-8. Similarly, in sites that do not bleed (BOP=No), a total of 62.73 ng/ml (out of the 280.0 ng/ml) is comprised of active MMP-8, and 217.27 ng/ml is latent MMP-8. In at least one embodiment, the MMP-8 distribution amount table 42 also contains a “TOTAL” column which represents a sum of all the active MMP-8 and latent MMP-8 from both bleeding and non-bleeding sites. Thus, in at least one embodiment, the data contained in the MMP-8 distribution amount table 42 allows the user application 28 and/or the medical service provider to view the data holistically or site-specifically in order to more accurately tailor a personalized treatment plan for the patient using the ratiometric measures discussed below.

In at least one embodiment, to accurately stratify patients based on risk of future disease and quantifying patient response to therapy, the user application 28 utilizes one or more select ratiometric measures governed by “enzymatic measures” data, which in turn are estimated and calculated by the user application 28 from the data described above. These ratiometric measures may be utilized both with respect to the whole mouth level 44 of data as well as the site-specific level 46 of data in order for the user application 28 to monitor changes in risk for a given patient. In at least one embodiment, various salient parameters may be utilized by the user application 28 in calculating different ratiometric measures for assessing inflammatory load and tissue destruction activity. In at least one embodiment, one such salient parameter used by the user application 28 is an enzyme ratio (320), which measures relative levels of active MMP-8 compared to latent MMP-8 in the patient's saliva. The enzyme ratio is calculated using the following formula:

$${Ratio}_{a / l} = ( \frac{a MMP - 8}{lMMP - 8} )$$

where aMMP-8 is the amount of active MMP-8 concentration and lMMP-8 is the amount of latent MMP-8 concentration in the patient's saliva. Accordingly, in the example illustrated in FIGS. 4-8, the enzyme ratio would be calculated as follows:

$$BOP = Yes : {Ratio}_{a / l}^{B O P = Y e s} = ( \frac{46.17}{65.83} ) = 0 \text{.70}$$

$$BOP = No : {Ratio}_{a / l}^{B O P = N o} = ( \frac{62.73}{217.27} ) = 0.29$$

$$Average : {Ratio}_{a / l}^{A v g} = ( \frac{108.9}{283.1} ) = 0.39$$

For sites that bleed, this means that approximately 70% of the 112.0 ng/ml MMP-8 load is in the form of active MMP-8 and 30% is in the form of latent MMP-8 in the patient's saliva during that visit. And since active enzyme is believed to be responsible for soft and hard tissue destruction, this would be a clinically worrisome outcome if it continues. The goal is to reduce this burden to below 50%. In contrast, for sites that do not bleed, approximately 30% of the 280.0 ng/ml MMP-8 load is in the form of active MMP-8 and 70% is in the form of latent MMP-8, which is relatively better.

In at least one embodiment, another such salient parameter used by the user application 28 is an oral inflammatory burden (“OIB”) (322). As periodontitis is an inflammatory response to a biological insult—microbial dysbiosis below the gum lines—the process will lead to increased secretion of pro-inflammatory cytokines such as interleukin IL-1α, IL-1β, IL-6, and tumor necrosis factor-α (TNF-α). Following this, neutrophils release a variety of enzymes such as MMP-8. In a healthy mouth, almost all of it is found in the latent form. As gingivitis and periodontal disease sets in, the tissue processes the latent form into active MMP-8, which starts to degrade soft and hard tissue. The degree of this activation depends on the level of inflammation and pathogenic microbial dysbiosis or insult. As such, the level of total MMP-8 in saliva (or “GCF”) can serve as an accurate proxy reflecting the level of whole mouth or site-specific inflammation. Thus, the oral inflammatory burden is the level of total MMP-8 in the patient's saliva. Due to multiple confounding variables in the biology of individuals, different patients can have widely differing levels of total MMP-8. For this reason, OIB alone should not be used to assess current or future risk. OIB should be used in conjunction with another parameter such as disease activity, discussed in detail below.

In at least one embodiment, another such salient parameter used by the user application 28 is disease activity (“DA”) (324), which measures relative levels of active MMP-8 compared to total MMP-8 in the patient's saliva. The disease activity is calculated using the following formula:

$$D A = ( \frac{a MMP - 8}{t MMP - 8} )$$

where aMMP-8 is the amount of active MMP-8 concentration in the patient's saliva and tMMP-8 is the amount of total MMP-8 concentration in the patient's saliva. DA is unitless and represents the relative percentage of active MMP-8 in the pool of total MMP-8. Thus, DA can have values ranging from 0 to 1; where 0 represents 0% active MMP-8 (or 100% latent MMP-8) and 1 represents the opposite spectrum, with 100% active MMP-8 (or 0% latent MMP-8). Accordingly, in the example illustrated in FIGS. 4-8, the DA values for average, bleeding (BOP=Yes) and non-bleeding (BOP=No) sites would be calculated as follows:

$$BOP = Yes : D {A}^{B O P = Y e s} = ( \frac{46.17}{46.17 + 65.83} ) = ( \frac{46.17}{112.} ) = 0 \text{.41}$$

$$BOP = No : D {A}^{B O P = N o} = ( \frac{62.73}{62.73 + 217.27} ) = ( \frac{62.73}{280.} ) = 0.22$$

$$Average : D {A}^{Avg .} = ( \frac{108.9}{283.1} ) = 0.28$$

This means that there is approximately 41% (0.41 from a scale of 0 to 1) of disease activity (as per enzyme measure) in sites that bleed compared to approximately 22% in sites that do not. On average, the patient has 28% of disease activity, as illustrated in FIG. 8.

Referring again to FIGS. 2 and 3, in at least one embodiment, depending on clinical status of periodontal disease in the patient (based on the PPD, BOP, OIB and DA values processed by the user application 28) during the patient's first visit, the medical service provider can prescribe and initiate an appropriate periodontal treatment (202) in an attempt to remove calculus and lower the pathogenic microbial burden behind periodontal disease.

In at least one embodiment, another salient parameter used by the user application 28 is a rate of change for each data point per unit of time, which allows standardization of data to a single common metric (i.e., time), simplifying patient risk stratification. For example, if two patients exhibit the same change in MMP-8 levels between two visits, but the time between visits is twice as long for a second one of the patients as compared to a first one of the patients, one would not ascribe the same risk to both patients. Instead, the first patient would be at higher risk as compared to the second patient, due to the relatively slower rate of change in second patient. Accordingly, in at least one embodiment, the user application 28 performs each of the above discussed calculations for the patient's saliva (302)-(324) that is collected during each subsequent visit and calculates a delta as well as an average value per day for each of the above discussed calculations (206). While single visit data points can provide a valuable glimpse into a patient's oral/periodontal status, the significance and sensitivity of the methods performed by the user application 28 using each of the above discussed calculations for the patient's saliva lies in the longitudinal dataset collected, both at a patient level as well as a public health level.

Accordingly, and by way of example, where an average whole mouth PPD value decreases from a first visit to a second visit in a span of 96 days, the user application 28 is able to conclude that the periodontal therapy initiated during the first visit has been effective. Additionally, a calculated rate of change of PPD per day is reflective of how fast the PPD value changed in the patient and is indicative of the inherent biology of tissue healing in the patient. On the other hand, where the calculated rate of change in the average whole mouth PPD has increased, the user application 28 is able to conclude that the periodontal therapy initiated during the first visit has not been effective. As another example, where a calculated rate of change in the enzyme ratio from a first visit to a second visit is negative, the user application 28 is able to conclude that the negative value and corresponding magnitude of the change reflects a decreased future risk for the patient. On the other hand, where the calculated rate of change in the enzyme ratio from a first visit to a second visit is positive, the user application 28 is able to conclude that the positive value and corresponding magnitude of the change reflects an increased future risk for the patient. As yet another example, where a calculated rate of change in the oral inflammatory burden from a first visit to a second visit is negative, the user application 28 is able to conclude that the oral inflammatory burden has decreased. On the other hand, where the calculated rate of change in the oral inflammatory burden from a first visit to a second visit is positive, the user application 28 is able to conclude that the oral inflammatory burden has increased. As yet another example, where a calculated rate of change in the disease activity from a first visit to a second visit is negative, the user application 28 is able to conclude that the progression of tissue destruction has decreased. On the other hand, where the calculated rate of change in the disease activity from a first visit to a second visit is positive, the user application 28 is able to conclude that the progression of tissue destruction has increased.

In at least one embodiment, another such salient parameter used by the user application 28 is fold change disease activity (“FCDA”), which represents a ratio in the magnitude of oral inflammatory burden, PPD and/or disease activity between visits—not just the delta. In at least one embodiment, the user application 28 calculates the FCDA status for one or more of the oral inflammatory burden, PPD and disease activity by dividing the ratios of each of these data points between visits by the time difference.

Thus, in at least one embodiment, the process of performing a saliva-based diagnostic screening on the patient (in order to assess periodontal risk, assess disease progression, predict future outcome and personalize patient treatment planning) further involves subsequent visits by the patient (204) through which the above-discussed steps (302)-(324) are performed during each subsequent visit. In at least one embodiment, a preliminary subsequent visit is made by the patient within one week of the first visit, during which steps (302), (304) and (320)-(324) are performed (i.e., no further PPD or BOP measurements are obtained during the preliminary subsequent visit). In such embodiments, the first visit represents a “before treatment” visit and the preliminary subsequent visit represents an “after treatment” visit, with the window of time between said visits representing an upper bound and a lower bound of the patient's effective response window (“ERW”) to the periodontal treatment performed at step (202). As discussed further below, longitudinal collection of PPD, BOP, enzyme ratio, OIB and DA values during further subsequent visits allows the user application 28 and the associated medical service provider to assess the rate of change in the data as it pertains to the patient, facilitating personalized planning. In at least one embodiment, these assessments are performed based on the rate of change of at least one of PPD, enzyme ratio, OIB and DA between visits. In at least one embodiment, the amount of time between visits is determined by the medical service provider based on the patient's biology and clinical history.

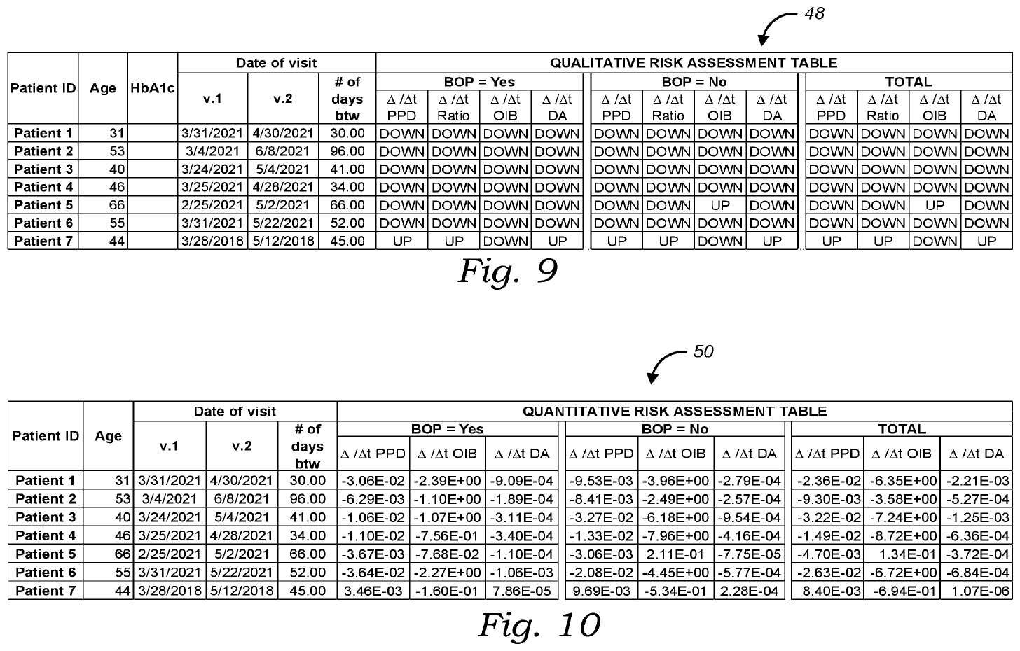

As noted above, in at least one embodiment, during each subsequent visit (not including the preliminary subsequent visit discussed above), steps (302)-(324) are performed. The user application 28 then populates at least one of a qualitative risk stratification table 48 (as illustrated in FIG. 9) and a quantitative risk stratification table 50 (as illustrated in FIG. 10) for the patient based on the calculated rate of change for each of the PPD, OIB and DA between the patient's latest visit and the patient's previous visit (208). In at least one embodiment, the values used to populate the qualitative risk stratification table 48 are “UP” (where the relevant data point value as measured during the patient's latest visit has increased since the patient's previous visit) and “DOWN” (where the relevant data point value as measured during the patient's latest visit has decreased since the patient's previous visit)—though in further embodiments, other values capable of qualitatively denoting an increasing or decreasing change could be substituted. In at least one embodiment, the values used to populate the quantitative risk stratification table 50 are the actual numerical values representing the rate of change for each of the PPD, OIB and DA between the patient's latest visit and the patient's previous visit. Thus, in such embodiments, the quantitative risk stratification table 50 is relatively more granular, as it incorporates both direction (i.e., increasing or decreasing data point values) as well as the respective magnitudes of such changes.

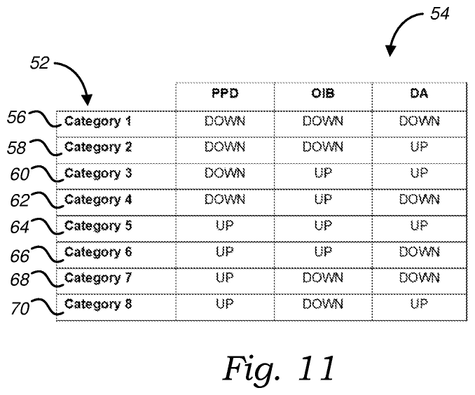

In at least one embodiment, with the qualitative and/or quantitative risk stratification table 48 and/or 50 populated, the user application 28 is capable of categorizing the patient into one of 8 diagnostic categories 52 based on a diagnostic table 54, as illustrated in FIG. 11. In at least one embodiment, the user application 28 categorizes the patient into a first category 56 upon determining that the PPD value has decreased as of the patient's latest visit, the OIB value has decreased as of the patient's latest visit, and the DA value has decreased as of the patient's latest visit. In at least one such embodiment, being categorized into the first category 56 means that the patient is at a relatively lower risk of experiencing a future adverse outcome. In a bit more detail, both the OIB and DA values are reduced, which indicates that the prescribed periodontal treatment has been effective in lowering all markers of disease burden. The OIB value indicates relative inflammation load is less as of the patent's latest visit as compared to the patient's previous visit and relative levels of active MMP-8 is less as of the patent's latest visit as compared to the patient's previous visit. The decrease in PPD values also means the patient's pocket depth is normalizing.

In at least one embodiment, the user application 28 categorizes the patient into a second category 58 upon determining that the PPD value has decreased as of the patient's latest visit, the OIB value has decreased as of the patient's latest visit, and the DA value has increased as of the patient's latest visit. In at least one such embodiment, being categorized into the second category 58 means that the patient is at a relatively higher risk of experiencing a future adverse outcome. In a bit more detail, while the OIB value has decreased, the DA value indicates an increased trend, which indicates that the prescribed periodontal treatment has been effective in lowering the OIB, but not by enough to nudge or change the intrinsic biology in the patient (as measured by DA) by much. There is an underlying signal (maybe microbial pathology at certain sites) that is keeping the MMP-8 activity, and thus tissue destruction, high. Additionally, the increasing DA value suggests that the relative levels of active MMP-8 has increased as of the patent's latest visit as compared to the patient's previous visit. The decrease in PPD values also means the patient's pocket depth is normalizing despite DA levels (i.e., tissue destruction) trending upward. This contradiction requires a more careful look at MMP-8 levels by the medical service provider. While an upward trend in DA values can be concerning, in the context of low MMP-8 levels, it may not pose much of a near-term future risk. For example, there may not be enough active MMP-8 in the collected saliva that resulted in measurable tissue destruction between the patient's visits. Given enough time, MMP-8 may accumulate to levels that will result in measurable clinical destruction.

In at least one embodiment, the user application 28 categorizes the patient into a third category 60 upon determining that the PPD value has decreased as of the patient's latest visit, the OIB value has increased as of the patient's latest visit, and the DA value has increased as of the patient's latest visit. In at least one such embodiment, being categorized into the third category 60 means that the patient is at a high risk of experiencing a future adverse outcome. In a bit more detail, both the OIB and DA values have increased, which indicates that the prescribed periodontal treatment has not been effective in lowering markers of disease activity. Additionally, the OIB value indicates that the relative inflammation load is greater as of the patent's latest visit as compared to the patient's previous visit, and relative levels of active MMP-8 are also higher as of the patent's latest visit as compared to the patient's previous visit. Of all three measured data points, only the PPD value has trended lower, which in the context of increasing OIB and DA values, it could indicate that the process of tissue destruction may have started recently without adequate time for it to be meaningfully recorded.

In at least one embodiment, the user application 28 categorizes the patient into a fourth category 62 upon determining that the PPD value has decreased as of the patient's latest visit, the OIB value has increased as of the patient's latest visit, and the DA value has decreased as of the patient's latest visit. In at least one such embodiment, being categorized into the fourth category 62 means that the patient is at a relatively lower risk of experiencing a near-term future adverse outcome. In a bit more detail, both the PPD and DA values have decreased, which indicates that the prescribed periodontal treatment has been effective in lowering markers of disease activity. However, the increased OIB value indicates an increase in inflammatory load. What is unknown is how the OIB will trend in the context of DA and relative levels of enzyme activation in the OIB. If the DA value exhibits an upward trend, then much of the increased OIB is being turned into active enzyme indicating an underlying biology that had not caught up as of the patient's latest visit. A follow-up MMP-8 testing will be warranted to determine the DA and OIB values in this context to accurately stratify the patient's risk.

In at least one embodiment, the user application 28 categorizes the patient into a fifth category 64 upon determining that the PPD value has increased as of the patient's latest visit, the OIB value has increased as of the patient's latest visit, and the DA value has increased as of the patient's latest visit. In at least one such embodiment, being categorized into the fifth category 64 means that the patient is at high risk of experiencing a future adverse outcome. In a bit more detail, the increase in all three data points indicates that the prescribed periodontal treatment has not been effective in reducing any of the markers. The patient may represent a population that is refractory to most periodontal treatments. Genetic predisposition may be at play. Also, more targeted therapies such site-specific antibiotic treatment may be warranted to keep the DA value at bay.

In at least one embodiment, the user application 28 categorizes the patient into a sixth category 66 upon determining that the PPD value has increased as of the patient's latest visit, the OIB value has increased as of the patient's latest visit, and the DA value has decreased as of the patient's latest visit. In at least one such embodiment, being categorized into the sixth category 66 means that the patient is potentially at a relatively lower risk of experiencing a near-term future adverse outcome. In a bit more detail, the upward trend of the OIB value offers a clue that over the longer term, the patient may have a high risk of experiencing a future adverse outcome as some of the latent MMP-8 converts into active MMP-8 and accelerates tissue destruction, resulting in DA reversing trends from decreasing to increasing values. In the context of increasing OIB values, the PPD value's upward trend is indicative that tissue destruction has not stopped and continues (which is also reflected in the OIB value increase). It is best to monitor the patient with another follow-up visit over a relatively shorter term to monitor and measure the change.

In at least one embodiment, the user application 28 categorizes the patient into a seventh category 68 upon determining that the PPD value has increased as of the patient's latest visit, the OIB value has decreased as of the patient's latest visit, and the DA value has decreased as of the patient's latest visit. In at least one such embodiment, being categorized into the seventh category 68 means that the patient is potentially at a relatively lower risk of experiencing a future adverse outcome. In a bit more detail, both the OIB and DA values have decreased, which indicates that the prescribed periodontal treatment has been effective in lowering the enzymatic markers of disease activity. Additionally, the OIB value indicates that the relative inflammation load is less as of the patent's latest visit as compared to the patient's previous visit, and relative levels of active MMP-8 is also lower as of the patent's latest visit as compared to the patient's previous visit. In the context of the decreasing OIB value, the increasing PPD value may indicate that tissue destruction is slow, possibly due to low levels of MMP-8 or slow healing of tissue in the patient or that tissue healing may have started recently without adequate time for this change to be meaningfully recorded.

In at least one embodiment, the user application 28 categorizes the patient into an eighth category 70 upon determining that the PPD value has increased as of the patient's latest visit, the OIB value has decreased as of the patient's latest visit, and the DA value has increased as of the patient's latest visit. In at least one such embodiment, being categorized into the eighth category 70 means that the patient has a moderate risk of experiencing a near-term future adverse outcome. In a bit more detail, unlike the fifth category 64, the risk in the eighth category 70 is moderate due to the decreasing OIB value. This means that despite the DA value being high as of the patient's latest visit, the lower OIB value translates to less inflammatory load and thus less MMP-8 levels, which suggests less pool of enzyme available to be activated, resulting in less tissue destruction. A follow-up test will allow for more accurate stratification of the patient's risk. It should be noted that these categories may be rearranged and reordered in further embodiments.

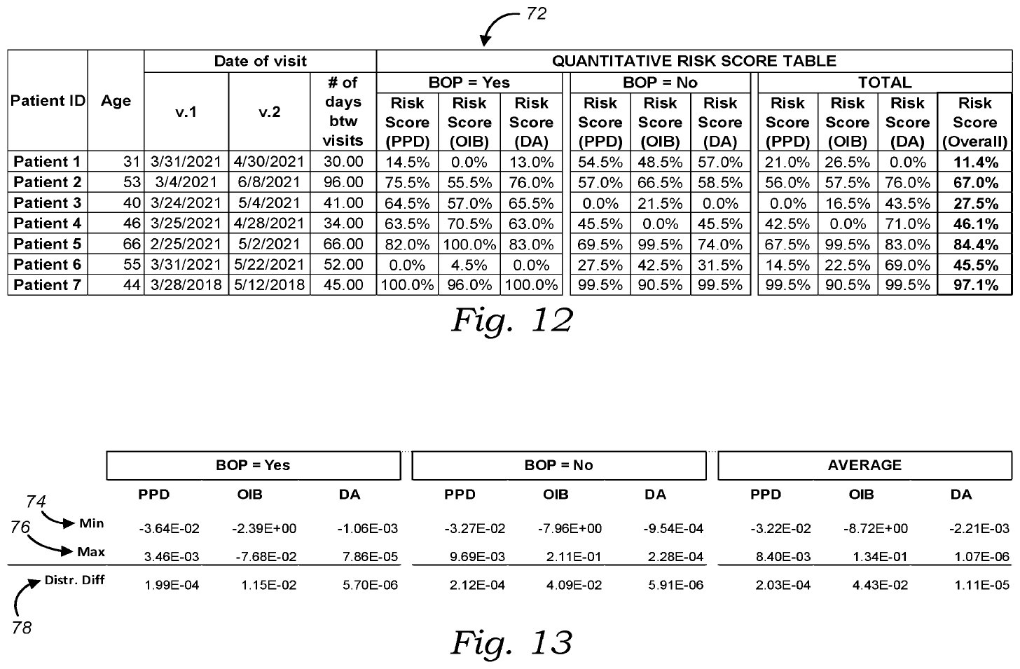

In at least one embodiment, where the user application 28 populates a quantitative risk stratification table 50 for the patient based on the calculated rate of change for each of the PPD, OIB and DA between the patient's latest visit and the patient's previous visit (208), the user application 28 further populates a quantitative risk score table 72 (as illustrated in FIG. 12) for the patient. In at least one embodiment, as illustrated in the table of FIG. 13, the user application 28 determines a minimum rate of change value 74 and a maximum rate of change value 76 for each of the PPD, OIB and DA based on all such rate of change values for all patients (or, alternatively, a select subset of all patients) stored in or otherwise accessible to the computing device 22 and calculates a distribution difference 78 for each of the PPD, OIB and DA by subtracting the corresponding minimum rate of change value from the corresponding maximum rate of change value, and dividing the difference by a desired interval quantity (such as 200, for example). The greater the interval quantity, the more granular the risk distribution will be between the corresponding minimum and maximum rate of change values, as discussed further below.

In at least one embodiment, the user application 28 next calculates a risk score (FIG. 12) for each value in the quantitative risk stratification table 50 using the following formula:

$$Risk Score ( x ) = \frac{x - minimum value}{maximum value - minimum value} * 100$$

Where x is a given rate of change value in the quantitative risk stratification table 50 for the patient, minimum value is the lowest rate of change value 74 for the corresponding one of the PPD, OIB and DA from all patients (or, alternatively, a select subset of all patients) stored in or otherwise accessible to the computing device 22, and maximum value is the highest rate of change value 76 for the corresponding one of the PPD, OIB and DA from all patients (or, alternatively, a select subset of all patients) stored in or otherwise accessible to the computing device 22. The calculated risk score is then added to the appropriate location in the quantitative risk score table 72. In at least one further embodiment, the user application 28 populates a risk distribution score table containing a risk score for each possible rate of change value for each of the PPD, OIB and DA, such that the subsequent determination of risk scores for particular rate of change values simply involves the user application 28 accessing the risk distribution score table in a lookup fashion.

Thus, in at least one such embodiment, use of the qualitative and/or quantitative risk stratification table 50 provides numerous benefits. Specifically, use of the qualitative and/or quantitative risk stratification table 48 and/or 50 avoids reliance on less accurate absolute levels of MMP-8 to classify each individual patient; it can accurately and objectively predict future risk based on biochemical markers (e.g., MMP-8, etc.), before clinical signs of disease become visually apparent; and it can also flag potential issues associated with subjective clinical measurements. There is currently no way for known prior art systems or methods to objectively assess a medical service provider's skills and motives in overestimating or underestimating PPD and/or BOP measures. This is especially true in real world practice.

Accordingly, the system 20 and associated methods are capable of predicting site specific levels within the patient's mouth using a single saliva sample collection during each of the patient's visits, which saves time, money and provides an effective solution for a proper clinical diagnostic workflow and workup. The system 20 not only provides the medical service provider with a relatively simple tool to assist with monitoring disease progression in real-time, but also provides payers with the data necessary to make payment decisions based on clinical outcomes. It should be noted that while the above-described methods are discussed in terms of the user application 28 carrying out one or more of the steps in said methods, in further embodiments, one or more of those steps may be carried out by the medical service provider instead, with or without the user application 28. Thus, the above-described methods should not be read as being reliant upon the user application 28 or even the computing device 22 in every embodiment.

Aspects of the present specification may also be described as the following embodiments:

1. A method for screening a volume of saliva of a patient for a presence or absence of active periodontal disease, the method comprising the steps of: implementing a user application residing in memory on an at least one computing device, the at least one computing device configured for receiving and processing select data, obtained by an at least one saliva screening device, based on the patient's saliva; and for each of an at least one patient visit to a medical service provider of the patient: collecting a volume of the patient's saliva; the user application determining a quantity of total MMP-8 along with a quantity of at least one of active MMP-8 and latent MMP-8 present in the collected saliva; the user application populating a clinical measurement table containing a plurality of probing pocket depth (“PPD”) values and corresponding bleeding on probing (“BOP”) values for an at least one site within a mouth of the patient as measured by a medical service provider for the patient; the user application populating a clinical distribution table containing a distribution of a quantity of BOP sites relative to each PPD value in the clinical measurement table; the user application calculating a weighted average PPD for each site where bleeding on probing is detected using the formula

$$f ( x ) = {\sum }_{x = 1}^{14} ( PP D ( x ) \times BOP Sites ( x ) ) / {\sum }_{x = 1}^{14} BOP Sites ( x )$$

where x is the PPD value corresponding to each site; the user application calculating a weighted average PPD for each site where bleeding on probing is not detected using the formula

$$f ( x ) = {\sum }_{x = 1}^{14} ( PP D ( x ) \times noBOP Sites ( x ) ) / {\sum }_{n = 1}^{14} noBOP Sites ( x )$$

where x is the PPD value corresponding to each site; the user application calculating a distribution of total MMP-8 quantities for each site where bleeding on probing is detected using the formula

$$f ( x ) = ( \frac{BOP Sites ( x )}{total sites} ) \times ( tMMP - 8 concentration )$$

where x is the PPD value corresponding to each site; the user application calculating a distribution of total MMP-8 quantities for each site where bleeding on probing is not detected using the formula

$$f ( x ) = ( \frac{noBOP Sites ( x )}{total sites} ) \times ( tMMP - 8 concentration )$$

where x is the PPD value corresponding to each site; the user application populating a total MMP-8 level table containing the calculated weighted average PPD and distribution of total MMP-8 quantities for each site; the user application populating an MMP-8 distribution percentage table containing a distribution percentage of active MMP-8 and a distribution percentage of latent MMP-8 for each site; the user application calculating an oral inflammatory burden (“OIB”) based on the level of total MMP-8 in the collected saliva; the user application calculating a disease activity (“DA”), representing a relative level of active MMP-8 compared to the level of total MMP-8 in the collected saliva, using the formula

$$D A = ( \frac{a MMP - 8}{t MMP - 8} )$$

where aMMP-8 is the amount of active MMP-8 concentration in the collected saliva and tMMP-8 is the amount of total MMP-8 concentration in the collected saliva; the user application calculating a rate of change for each of the PPD, OIB and DA values between the current patient visit and a previous patient visit; and the user application populating at least one of a qualitative risk stratification table and a quantitative risk stratification table based on the calculated rate of change for each of the PPD, OIB and DA values between the current patient visit and a previous patient visit; whereby, one or both of the qualitative risk stratification table and quantitative risk stratification table can be used by the medical service provider to accurately and objectively predict the patient's future risk of periodontal disease.

2. The method according to embodiment 1, wherein the step of the user application determining a quantity of total MMP-8 along with a quantity of at least one of active MMP-8 and latent MMP8, further comprises the steps of: the user application determining a quantity of an at least one ubiquitous protein present in the collected saliva; and the user application calculating a specific activity of at least one of the total MMP-8, active MMP-8 and latent MMP-8 using the formula

$$Specific Activity = \frac{MMP - 8}{ubiquitous protein} .$$

3. The method according to embodiments 1-2, wherein the step of the user application determining a quantity of total MMP-8 along with a quantity of at least one of active MMP-8 and latent MMP8, further comprises the step of analyzing the collected saliva using an enzyme-linked immunosorbent assay test.

4. The method according to embodiments 1-3, wherein the step of the user application determining a quantity of total MMP-8 along with a quantity of at least one of active MMP-8 and latent MMP8, further comprises the step of analyzing the collected saliva using a lateral flow assay test.

5. The method according to embodiments 1-4, wherein the step of collecting a volume of the patient's saliva further comprises the step of collecting a volume of the patient's saliva prior to performing any treatments or clinical measurements on the patient.

6. The method according to embodiments 1-5, wherein the step of the user application calculating a disease activity further comprises the steps of: the user application calculating a disease activity for sites where bleeding on probing is detected; and the user application calculating a disease activity for sites where bleeding on probing is not detected.

7. The method according to embodiments 1-6, wherein the step of the user application calculating a rate of change for each of the PPD, OIB and DA values between the current patient visit and a previous patient visit further comprises the steps of: the user application calculating a rate of change for each of the PPD, OIB and DA values for sites where bleeding on probing is detected; and the user application calculating a rate of change for each of the PPD, OIB and DA values for sites where bleeding on probing is not detected.

8. The method according to embodiments 1-7, further comprising the step of the user application calculating an enzyme ratio, representing relative levels of active MMP-8 compared to latent MMP-8 in the collected saliva, using the formula

$${Ratio}_{a / l} = ( \frac{a MMP - 8}{lMMP - 8} )$$

where aMMP-8 is the amount of active MMP-8 concentration in the collected saliva, and lMMP-8 is the amount of latent MMP-8 concentration in the collected saliva.

9. The method according to embodiments 1-8, wherein the step of the user application calculating an enzyme ratio further comprises the steps of: the user application calculating an enzyme ratio for sites where bleeding on probing is detected; and the user application calculating an enzyme ratio for sites where bleeding on probing is not detected.

10. The method according to embodiments 1-9, further comprising the step of the user application calculating a rate of change for the enzyme ratio values between the current patient visit and a previous patient visit.

11. The method according to embodiments 1-10, wherein the step of the user application calculating a rate of change for the enzyme ratio values between the current patient visit and a previous patient visit further comprises the steps of: the user application calculating a rate of change for the enzyme ratio values for sites where bleeding on probing is detected; and the user application calculating a rate of change for the enzyme ratio values for sites where bleeding on probing is not detected.

12. The method according to embodiments 1-11, further comprising the step of the user application calculating a fold change disease activity (“FCDA”) representing a ratio in a magnitude of at least one of the OIB, PPD and DA value between the current patient visit and a previous patient visit, with said ratio divided by an amount of elapsed time between the current patient visit and the previous patient visit.

13. The method according to embodiments 1-12, wherein the step of the user application populating at least one of a qualitative risk stratification table and a quantitative risk stratification table further comprises the step of the user application populating a qualitative risk stratification table containing indicators denoting respective increases and decreases in the calculated rate of change for each of the PPD, OIB and DA values between the current patient visit and a previous patient visit.

14. The method according to embodiments 1-13, wherein the step of the user application populating at least one of a qualitative risk stratification table and a quantitative risk stratification table further comprises the step of the user application populating a quantitative risk stratification table containing the calculated rate of change for each of the PPD, OIB and DA values between the current patient visit and a previous patient visit.

15. The method according to embodiments 1-14, further comprising the step of the user application populating a quantitative risk score table containing a risk score for each rate of change value in the quantitative risk stratification table.

16. The method according to embodiments 1-15, further comprising the step of the user application calculating the risk score for each rate of change value in the quantitative risk stratification table by, for each of rate of change value in the quantitative risk stratification table: the user application determining a minimum rate of change value for the corresponding one of the PPD, OIB and DA values based on all such rate of change values for all patients; the user application determining a maximum rate of change value for the corresponding one of the PPD, OIB and DA values based on all such rate of change values for all patients; and the user application calculating the risk score using the formula

$$Risk Score ( x ) = \frac{x - minimum value}{maximum value - minimum value} * 100$$

where x is the rate of change value in the quantitative risk stratification table for the patient, minimum value is the lowest rate of change value from all patients, and maximum value is the highest rate of change value from all patients.

17. The method according to embodiments 1-16, further comprising the step of the user application, for each of rate of change value in the quantitative risk stratification table, calculating a distribution difference for the corresponding one of the PPD, OIB and DA values by subtracting the corresponding minimum rate of change value from the corresponding maximum rate of change value, and dividing the difference by a desired interval quantity.

18. The method according to embodiments 1-17, further comprising the steps of: the user application categorizing the patient into a first category upon determining that the PPD value has decreased, the OIB value has decreased, and the DA value has decreased between the current patient visit and a previous patient visit, said first category indicative of the patient being at a relatively lower risk of experiencing a future adverse outcome; the user application categorizing the patient into a second category upon determining that the PPD value has decreased, the OIB value has decreased, and the DA value has increased between the current patient visit and a previous patient visit, said second category indicative of the patient being at a relatively higher risk of experiencing a future adverse outcome; the user application categorizing the patient into a third category upon determining that the PPD value has decreased, the OIB value has increased, and the DA value has increased between the current patient visit and a previous patient visit, said third category indicative of the patient being at a high risk of experiencing a future adverse outcome; the user application categorizing the patient into a fourth category upon determining that the PPD value has decreased, the OIB value has increased, and the DA value has decreased between the current patient visit and a previous patient visit, said fourth category indicative of the patient being at a relatively lower risk of experiencing a near-term future adverse outcome; the user application categorizing the patient into a fifth category upon determining that the PPD value has increased, the OIB value has increased, and the DA value has increased between the current patient visit and a previous patient visit, said fifth category indicative of the patient being at high risk of experiencing a future adverse outcome; the user application categorizing the patient into a sixth category upon determining that the PPD value has increased, the OIB value has increased, and the DA value has decreased between the current patient visit and a previous patient visit, said sixth category indicative of the patient being at a relatively lower risk of experiencing a near-term future adverse outcome; the user application categorizing the patient into a seventh category upon determining that the PPD value has increased, the OIB value has decreased, and the DA value has decreased between the current patient visit and a previous patient visit, said seventh category indicative of the patient potentially being at a relatively lower risk of experiencing a future adverse outcome; and the user application categorizing the patient into an eighth category upon determining that the PPD value has increased, the OIB value has decreased, and the DA value has increased between the current patient visit and a previous patient visit, said eighth category indicative of the patient being at a moderate risk of experiencing a near-term future adverse outcome.

19. The method according to embodiments 1-18, further comprising the step of the user application populating an MMP-8 distribution amount table containing data from each of the clinical distribution table, total MMP-8 level table and MMP-8 distribution percentage table, the MMP-8 distribution amount table containing data for both a whole mouth level of data and a site-specific level of data.

20. The method according to embodiments 1-19, wherein the step of the user application populating an MMP-8 distribution percentage table further comprises the step of the user application calculating the distribution percentage of active MMP-8 and the distribution percentage of latent MMP-8 for each site using the normal regression curve formula

$$f ( x ) = \frac{{L}_{Max}}{( 1 + {e}^{- k ( x - {x}_{0} )} )}$$

where e is the natural logarithm base, x₀ is the x-value of the curve's midpoint, L_{Max} is the curve's maximum value, and k is the slope of the curve.

21. A saliva-based diagnostic screening system for screening a volume of saliva of a patient for a presence or absence of active periodontal disease, the system comprising: a user application residing in memory on an at least one computing device, the at least one computing device configured for receiving and processing select data, obtained by an at least one saliva screening device, based on the patient's saliva; wherein, for each of an at least one patient visit to a medical service provider of the patient, the user application is configured for: determining a quantity of total MMP-8 along with a quantity of at least one of active MMP-8 and latent MMP-8 present in a volume of saliva collected from the patient; populating a clinical measurement table containing a plurality of probing pocket depth (“PPD”) values and corresponding bleeding on probing (“BOP”) values for an at least one site within a mouth of the patient as measured by a medical service provider for the patient; populating a clinical distribution table containing a distribution of a quantity of BOP sites relative to each PPD value in the clinical measurement table; calculating a weighted average PPD for each site where bleeding on probing is detected using the formula

$$f ( x ) = {\sum }_{x = 1}^{14} ( PP D ( x ) \times BOP Sites ( x ) ) / {\sum }_{x = 1}^{14} BOP Sites ( x )$$

where x is the PPD value corresponding to each site; calculating a weighted average PPD for each site where bleeding on probing is not detected using the formula

$$f ( x ) = {\sum }_{x = 1}^{14} ( PP D ( x ) \times noBOP Sites ( x ) ) / {\sum }_{n = 1}^{14} noBOP Sites ( x )$$

where x is the PPD value corresponding to each site; calculating a distribution of total MMP-8 quantities for each site where bleeding on probing is detected using the formula

$$f ( x ) = ( \frac{BOP Sites ( x )}{total sites} ) \times ( tMMP - 8 concentration )$$

where x is the PPD value corresponding to each site; calculating a distribution of total MMP-8 quantities for each site where bleeding on probing is not detected using the formula

$$f ( x ) = ( \frac{noBOP Sites ( x )}{total sites} ) \times ( tMMP - 8 concentration )$$

where x is the PPD value corresponding to each site; populating a total MMP-8 level table containing the calculated weighted average PPD and distribution of total MMP-8 quantities for each site; populating a total MMP-8 distribution percentage table containing a distribution percentage of active MMP-8 and a distribution percentage of latent MMP-8 for each site based; calculating an oral inflammatory burden (“OIB”) based on the level of total MMP-8 in the collected saliva; calculating a disease activity (“DA”), representing a relative level of active MMP-8 compared to the level of total MMP-8 in the collected saliva, using the formula

$$D A = ( \frac{a MMP - 8}{t MMP - 8} )$$

where aMMP-8 is the amount of active MMP-8 concentration in the collected saliva and tMMP-8 is the amount of total MMP-8 concentration in the collected saliva; calculating a rate of change for each of the PPD, OIB and DA values between the current patient visit and a previous patient visit; and populating at least one of a qualitative risk stratification table and a quantitative risk stratification table based on the calculated rate of change for each of the PPD, OIB and DA values between the current patient visit and a previous patient visit; whereby, one or both of the qualitative risk stratification table and quantitative risk stratification table can be used by the medical service provider to accurately and objectively predict the patient's future risk of periodontal disease.

22. The saliva-based diagnostic screening system according to embodiment 21, wherein while determining a quantity of total MMP-8 along with a quantity of at least one of active MMP-8 and latent MMP8, the user application is further configured for: determining a quantity of an at least one ubiquitous protein present in the collected saliva; and calculating a specific activity of at least one of the total MMP-8, active MMP-8 and latent MMP-8 using the formula

$$Specific Activity = \frac{MMP - 8}{ubiquitous protein} .$$

23. The saliva-based diagnostic screening system according to embodiments 21-22, wherein while determining a quantity of total MMP-8 along with a quantity of at least one of active MMP-8 and latent MMP8, the user application is further configured for analyzing the collected saliva using an enzyme-linked immunosorbent assay test.

24. The saliva-based diagnostic screening system according to embodiments 21-23, wherein while determining a quantity of total MMP-8 along with a quantity of at least one of active MMP-8 and latent MMP8, the user application is further configured for analyzing the collected saliva using a lateral flow assay test.

25. The saliva-based diagnostic screening system according to embodiments 21-24, wherein while calculating a disease activity, the user application is further configured for: calculating a disease activity for sites where bleeding on probing is detected; and calculating a disease activity for sites where bleeding on probing is not detected.

26. The saliva-based diagnostic screening system according to embodiments 21-25, wherein while calculating a rate of change for each of the PPD, OIB and DA values between the current patient visit and a previous patient visit, the user application is further configured for: calculating a rate of change for each of the PPD, OIB and DA values for sites where bleeding on probing is detected; and calculating a rate of change for each of the PPD, OIB and DA values for sites where bleeding on probing is not detected.

27. The saliva-based diagnostic screening system according to embodiments 21-26, wherein the user application is further configured for calculating an enzyme ratio, representing relative levels of active MMP-8 compared to latent MMP-8 in the collected saliva, using the formula

$${Ratio}_{a / l} = ( \frac{a MMP - 8}{lMMP - 8} )$$

where aMMP-8 is the amount of active MMP-8 concentration in the collected saliva, and lMMP-8 is the amount of latent MMP-8 concentration in the collected saliva.

28. The saliva-based diagnostic screening system according to embodiments 21-27, wherein while calculating an enzyme ratio, the user application is further configured for: calculating an enzyme ratio for sites where bleeding on probing is detected; and calculating an enzyme ratio for sites where bleeding on probing is not detected.

29. The saliva-based diagnostic screening system according to embodiments 21-28, wherein the user application is further configured for calculating a rate of change for the enzyme ratio values between the current patient visit and a previous patient visit.

30. The saliva-based diagnostic screening system according to embodiments 21-29, wherein while calculating a rate of change for the enzyme ratio values between the current patient visit and a previous patient visit, the user application is further configured for: calculating a rate of change for the enzyme ratio values for sites where bleeding on probing is detected; and calculating a rate of change for the enzyme ratio values for sites where bleeding on probing is not detected.

31. The saliva-based diagnostic screening system according to embodiments 21-30, wherein the user application is further configured for calculating a fold change disease activity (“FCDA”) representing a ratio in a magnitude of at least one of the OIB, PPD and DA value between the current patient visit and a previous patient visit, with said ratio divided by an amount of elapsed time between the current patient visit and the previous patient visit.

32. The saliva-based diagnostic screening system according to embodiments 21-31, wherein while populating at least one of a qualitative risk stratification table and a quantitative risk stratification table, the user application is further configured for populating a qualitative risk stratification table containing indicators denoting respective increases and decreases in the calculated rate of change for each of the PPD, OIB and DA values between the current patient visit and a previous patient visit.

33. The saliva-based diagnostic screening system according to embodiments 21-32, wherein while populating at least one of a qualitative risk stratification table and a quantitative risk stratification table, the user application is further configured for populating a quantitative risk stratification table containing the calculated rate of change for each of the PPD, OIB and DA values between the current patient visit and a previous patient visit.

34. The saliva-based diagnostic screening system according to embodiments 21-33, wherein the user application is further configured for populating a quantitative risk score table containing a risk score for each rate of change value in the quantitative risk stratification table.

35. The saliva-based diagnostic screening system according to embodiments 21-34, wherein the user application is further configured for calculating the risk score for each rate of change value in the quantitative risk stratification table by, for each of rate of change value in the quantitative risk stratification table: determining a minimum rate of change value for the corresponding one of the PPD, OIB and DA values based on all such rate of change values for all patients; determining a maximum rate of change value for the corresponding one of the PPD, OIB and DA values based on all such rate of change values for all patients; and calculating the risk score using the formula

$$Risk Score ( x ) = \frac{x - minimum value}{maximum value - minimum value} * 100$$

where x is the rate of change value in the quantitative risk stratification table for the patient, minimum value is the lowest rate of change value from all patients, and maximum value is the highest rate of change value from all patients.

36. The saliva-based diagnostic screening system according to embodiments 21-35, wherein the user application is further configured for, for each of rate of change value in the quantitative risk stratification table, calculating a distribution difference for the corresponding one of the PPD, OIB and DA values by subtracting the corresponding minimum rate of change value from the corresponding maximum rate of change value, and dividing the difference by a desired interval quantity.

37. The saliva-based diagnostic screening system according to embodiments 21-36, wherein the user application is further configured for: categorizing the patient into a first category upon determining that the PPD value has decreased, the OIB value has decreased, and the DA value has decreased between the current patient visit and a previous patient visit, said first category indicative of the patient being at a relatively lower risk of experiencing a future adverse outcome; categorizing the patient into a second category upon determining that the PPD value has decreased, the OIB value has decreased, and the DA value has increased between the current patient visit and a previous patient visit, said second category indicative of the patient being at a relatively higher risk of experiencing a future adverse outcome; categorizing the patient into a third category upon determining that the PPD value has decreased, the OIB value has increased, and the DA value has increased between the current patient visit and a previous patient visit, said third category indicative of the patient being at a high risk of experiencing a future adverse outcome; categorizing the patient into a fourth category upon determining that the PPD value has decreased, the OIB value has increased, and the DA value has decreased between the current patient visit and a previous patient visit, said fourth category indicative of the patient being at a relatively lower risk of experiencing a near-term future adverse outcome; categorizing the patient into a fifth category upon determining that the PPD value has increased, the OIB value has increased, and the DA value has increased between the current patient visit and a previous patient visit, said fifth category indicative of the patient being at high risk of experiencing a future adverse outcome; categorizing the patient into a sixth category upon determining that the PPD value has increased, the OIB value has increased, and the DA value has decreased between the current patient visit and a previous patient visit, said sixth category indicative of the patient being at a relatively lower risk of experiencing a near-term future adverse outcome; categorizing the patient into a seventh category upon determining that the PPD value has increased, the OIB value has decreased, and the DA value has decreased between the current patient visit and a previous patient visit, said seventh category indicative of the patient potentially being at a relatively lower risk of experiencing a future adverse outcome; and categorizing the patient into an eighth category upon determining that the PPD value has increased, the OIB value has decreased, and the DA value has increased between the current patient visit and a previous patient visit, said eighth category indicative of the patient being at a moderate risk of experiencing a near-term future adverse outcome.

38. The saliva-based diagnostic screening system according to embodiments 21-37, wherein the user application is further configured for populating an MMP-8 distribution amount table containing data from each of the clinical distribution table, total MMP-8 level table and MMP-8 distribution percentage table, the MMP-8 distribution amount table containing data for both a whole mouth level of data and a site-specific level of data.

39. The saliva-based diagnostic screening system according to embodiments 21-38, further comprising an at least one data storage device in selective communication with the computing device and configured for storing said data obtained by the at least one saliva screening device and processed by the computing device.

40. The saliva-based diagnostic screening system according to embodiments 21-39, wherein while populating an MMP-8 distribution percentage table, the user application is further configured for calculating the distribution percentage of active MMP-8 and the distribution percentage of latent MMP-8 for each site using the normal regression curve formula

$$f ( x ) = \frac{{L}_{Max}}{( 1 + {e}^{- k ( x - {x}_{0} )} )}$$

where e is the natural logarithm base, x₀ is the x-value of the curve's midpoint, L_{Max} is the curve's maximum value, and k is the slope of the curve.

41. A non-transient computer readable medium containing program instructions for causing an at least one computing device to perform a method of screening a volume of saliva of a patient for a presence or absence of active periodontal disease, the method comprising the steps of, for each of an at least one patient visit to a medical service provider of the patient: determining a quantity of total MMP-8 along with a quantity of at least one of active MMP-8 and latent MMP-8 present in a volume of saliva collected from the patient; populating a clinical measurement table containing a plurality of probing pocket depth (“PPD”) values and corresponding bleeding on probing (“BOP”) values for an at least one site within a mouth of the patient as measured by a medical service provider for the patient; populating a clinical distribution table containing a distribution of a quantity of BOP sites relative to each PPD value in the clinical measurement table; calculating a weighted average PPD for each site where bleeding on probing is detected using the formula

$$f ( x ) = {\sum }_{x = 1}^{14} P P D ( x ) \times BOP Sites ( x ) / {\sum }_{x = 1}^{14} BOP Sites ( x )$$

where x is the PPD value corresponding to each site; calculating a weighted average PPD for each site where bleeding on probing is not detected using the formula

$$f ( x ) = {\sum }_{x = 1}^{14} ( PP D ( x ) \times BOP Sites ( x ) ) / {\sum }_{n = 1}^{14} noBOP Sites ( x )$$

where x is the PPD value corresponding to each site; calculating a distribution of total MMP-8 quantities for each site where bleeding on probing is detected using the formula

$$f ( x ) = ( \frac{BOP Sites ( x )}{total sites} ) \times ( tMMP - 8 concentration )$$

where x is the PPD value corresponding to each site; calculating a distribution of total MMP-8 quantities for each site where bleeding on probing is not detected using the formula

$$f ( x ) = ( \frac{noBOP Sites ( x )}{total sites} ) \times ( tMMP - 8 concentration )$$

where x is the PPD value corresponding to each site; populating a total MMP-8 level table containing the calculated weighted average PPD and distribution of total MMP-8 quantities for each site; populating an MMP-8 distribution percentage table containing a distribution percentage of active MMP-8 and a distribution percentage of latent MMP-8 for each site; calculating an oral inflammatory burden (“OIB”) based on the level of total MMP-8 in the collected saliva; calculating a disease activity (“DA”), representing a relative level of active MMP-8 compared to the level of total MMP-8 in the collected saliva, using the formula

$$D A = ( \frac{a MMP - 8}{t MMP - 8} )$$

where aMMP-8 is the amount of active MMP-8 concentration in the collected saliva and tMMP-8 is the amount of total MMP-8 concentration in the collected saliva; calculating a rate of change for each of the PPD, OIB and DA values between the current patient visit and a previous patient visit; and populating at least one of a qualitative risk stratification table and a quantitative risk stratification table based on the calculated rate of change for each of the PPD, OIB and DA values between the current patient visit and a previous patient visit; whereby, one or both of the qualitative risk stratification table and quantitative risk stratification table can be used by the medical service provider to accurately and objectively predict the patient's future risk of periodontal disease.

42. The method according to embodiment 41, wherein the step of determining a quantity of total MMP-8 along with a quantity of at least one of active MMP-8 and latent MMP8, further comprises the steps of: determining a quantity of an at least one ubiquitous protein present in the collected saliva; and calculating a specific activity of at least one of the total MMP-8, active MMP-8 and latent MMP-8 using the formula

$$Specific Activity = \frac{MMP - 8}{ubiquitous protein} .$$

43. The method according to embodiments 41-42, wherein the step of determining a quantity of total MMP-8 along with a quantity of at least one of active MMP-8 and latent MMP8, further comprises the step of analyzing the collected saliva using an enzyme-linked immunosorbent assay test.

44. The method according to embodiments 41-43, wherein the step of determining a quantity of total MMP-8 along with a quantity of at least one of active MMP-8 and latent MMP8, further comprises the step of analyzing the collected saliva using a lateral flow assay test.

45. The method according to embodiments 41-44, wherein the step of collecting a volume of the patient's saliva further comprises the step of collecting a volume of the patient's saliva prior to performing any treatments or clinical measurements on the patient.

46. The method according to embodiments 41-45, wherein the step of calculating a disease activity further comprises the steps of: calculating a disease activity for sites where bleeding on probing is detected; and calculating a disease activity for sites where bleeding on probing is not detected.

47. The method according to embodiments 41-46, wherein the step of calculating a rate of change for each of the PPD, OIB and DA values between the current patient visit and a previous patient visit further comprises the steps of: calculating a rate of change for each of the PPD, OIB and DA values for sites where bleeding on probing is detected; and calculating a rate of change for each of the PPD, OIB and DA values for sites where bleeding on probing is not detected.

48. The method according to embodiments 41-47, further comprising the step of calculating an enzyme ratio, representing relative levels of active MMP-8 compared to latent MMP-8 in the collected saliva, using the formula

$${Ratio}_{a / l} = ( \frac{a MMP - 8}{lMMP - 8} )$$

where aMMP-8 is the amount of active MMP-8 concentration in the collected saliva, and lMMP-8 is the amount of latent MMP-8 concentration in the collected saliva.

49. The method according to embodiments 41-48, wherein the step of calculating an enzyme ratio further comprises the steps of: calculating an enzyme ratio for sites where bleeding on probing is detected; and calculating an enzyme ratio for sites where bleeding on probing is not detected.

50. The method according to embodiments 41-49, further comprising the step of calculating a rate of change for the enzyme ratio values between the current patient visit and a previous patient visit.

51. The method according to embodiments 41-50, wherein the step of calculating a rate of change for the enzyme ratio values between the current patient visit and a previous patient visit further comprises the steps of: calculating a rate of change for the enzyme ratio values for sites where bleeding on probing is detected; and calculating a rate of change for the enzyme ratio values for sites where bleeding on probing is not detected.

52. The method according to embodiments 41-51, further comprising the step of calculating a fold change disease activity (“FCDA”) representing a ratio in a magnitude of at least one of the OIB, PPD and DA value between the current patient visit and a previous patient visit, with said ratio divided by an amount of elapsed time between the current patient visit and the previous patient visit.

53. The method according to embodiments 41-52, wherein the step of populating at least one of a qualitative risk stratification table and a quantitative risk stratification table further comprises the step of populating a qualitative risk stratification table containing indicators denoting respective increases and decreases in the calculated rate of change for each of the PPD, OIB and DA values between the current patient visit and a previous patient visit.

54. The method according to embodiments 41-53, wherein the step of populating at least one of a qualitative risk stratification table and a quantitative risk stratification table further comprises the step of populating a quantitative risk stratification table containing the calculated rate of change for each of the PPD, OIB and DA values between the current patient visit and a previous patient visit.

55. The method according to embodiments 41-54, further comprising the step of populating a quantitative risk score table containing a risk score for each rate of change value in the quantitative risk stratification table.

56. The method according to embodiments 41-55, further comprising the step of calculating the risk score for each rate of change value in the quantitative risk stratification table by, for each of rate of change value in the quantitative risk stratification table: determining a minimum rate of change value for the corresponding one of the PPD, OIB and DA values based on all such rate of change values for all patients; determining a maximum rate of change value for the corresponding one of the PPD, OIB and DA values based on all such rate of change values for all patients; and calculating the risk score using the formula

$$Risk Score ( x ) = \frac{x - minimum value}{maximum value - minimum value} * 100$$

where x is the rate of change value in the quantitative risk stratification table for the patient, minimum value is the lowest rate of change value from all patients, and maximum value is the highest rate of change value from all patients.

57. The method according to embodiments 41-56, further comprising the step of, for each of rate of change value in the quantitative risk stratification table, calculating a distribution difference for the corresponding one of the PPD, OIB and DA values by subtracting the corresponding minimum rate of change value from the corresponding maximum rate of change value, and dividing the difference by a desired interval quantity.

58. The method according to embodiments 41-57, further comprising the steps of: categorizing the patient into a first category upon determining that the PPD value has decreased, the OIB value has decreased, and the DA value has decreased between the current patient visit and a previous patient visit, said first category indicative of the patient being at a relatively lower risk of experiencing a future adverse outcome; categorizing the patient into a second category upon determining that the PPD value has decreased, the OIB value has decreased, and the DA value has increased between the current patient visit and a previous patient visit, said second category indicative of the patient being at a relatively higher risk of experiencing a future adverse outcome; categorizing the patient into a third category upon determining that the PPD value has decreased, the OIB value has increased, and the DA value has increased between the current patient visit and a previous patient visit, said third category indicative of the patient being at a high risk of experiencing a future adverse outcome; categorizing the patient into a fourth category upon determining that the PPD value has decreased, the OIB value has increased, and the DA value has decreased between the current patient visit and a previous patient visit, said fourth category indicative of the patient being at a relatively lower risk of experiencing a near-term future adverse outcome; categorizing the patient into a fifth category upon determining that the PPD value has increased, the OIB value has increased, and the DA value has increased between the current patient visit and a previous patient visit, said fifth category indicative of the patient being at high risk of experiencing a future adverse outcome; categorizing the patient into a sixth category upon determining that the PPD value has increased, the OIB value has increased, and the DA value has decreased between the current patient visit and a previous patient visit, said sixth category indicative of the patient being at a relatively lower risk of experiencing a near-term future adverse outcome; categorizing the patient into a seventh category upon determining that the PPD value has increased, the OIB value has decreased, and the DA value has decreased between the current patient visit and a previous patient visit, said seventh category indicative of the patient potentially being at a relatively lower risk of experiencing a future adverse outcome; and categorizing the patient into an eighth category upon determining that the PPD value has increased, the OIB value has decreased, and the DA value has increased between the current patient visit and a previous patient visit, said eighth category indicative of the patient being at a moderate risk of experiencing a near-term future adverse outcome.

59. The method according to embodiments 41-58, further comprising the step of populating an MMP-8 distribution amount table containing data from each of the clinical distribution table, total MMP-8 level table and MMP-8 distribution percentage table, the MMP-8 distribution amount table containing data for both a whole mouth level of data and a site-specific level of data.

60. The method according to embodiments 41-59, wherein the step of populating an MMP-8 distribution percentage table further comprises the step of calculating the distribution percentage of active MMP-8 and the distribution percentage of latent MMP-8 for each site using the normal regression curve formula

$$f ( x ) = \frac{{L}_{Max}}{( 1 + {e}^{- k ( x - {x}_{0} )} )}$$

where e is the natural logarithm base, x₀ is the x-value of the curve's midpoint, L_{Max} is the curve's maximum value, and k is the slope of the curve.

61. A method for screening a volume of saliva of a patient for a presence or absence of active periodontal disease, the method comprising the steps of, for each of an at least one patient visit to a medical service provider of the patient: collecting a volume of the patient's saliva; determining a quantity of total MMP-8 along with a quantity of at least one of active MMP-8 and latent MMP-8 present in the collected saliva; populating a clinical measurement table containing a plurality of probing pocket depth (“PPD”) values and corresponding bleeding on probing (“BOP”) values for an at least one site within a mouth of the patient as measured by a medical service provider for the patient; populating a clinical distribution table containing a distribution of a quantity of BOP sites relative to each PPD value in the clinical measurement table; calculating a weighted average PPD for each site where bleeding on probing is detected using the formula

$$f ( x ) = {\sum }_{x = 1}^{14} ( PPD ( x ) \times BOP Sites ( x ) ) / {\sum }_{x = 1}^{14} BOP Sites ( x )$$

where x is the PPD value corresponding to each site; calculating a weighted average PPD for each site where bleeding on probing is not detected using the formula

$$f ( x ) = {\sum }_{x = 1}^{14} ( PPD ( x ) \times noBOP Sites ( x ) ) / {\sum }_{x = 1}^{14} noBOP Sites ( x )$$

where x is the PPD value corresponding to each site; calculating a distribution of total MMP-8 quantities for each site where bleeding on probing is detected using the formula

$$f ( x ) = ( \frac{BOP Sites ( x )}{total sites} ) \times ( tMMP - 8 concentration )$$

where x is the PPD value corresponding to each site; calculating a distribution of total MMP-8 quantities for each site where bleeding on probing is not detected using the formula

$$f ( x ) = ( \frac{noBOP Sites ( x )}{total sites} ) \times ( tMMP - 8 concentration )$$

where x is the PPD value corresponding to each site; populating a total MMP-8 level table containing the calculated weighted average PPD and distribution of total MMP-8 quantities for each site; populating an MMP-8 distribution percentage table containing a distribution percentage of active MMP-8 and a distribution percentage of latent MMP-8 for each site; calculating an oral inflammatory burden (“OIB”) based on the level of total MMP-8 in the collected saliva; calculating a disease activity (“DA”), representing a relative level of active MMP-8 compared to the level of total MMP-8 in the collected saliva, using the formula

$$DA = ( \frac{aMMP - 8}{tMMP - 8} )$$

where aMMP-8 is the amount of active MMP-8 concentration in the collected saliva and tMMP-8 is the amount of total MMP-8 concentration in the collected saliva; calculating a rate of change for each of the PPD, OIB and DA values between the current patient visit and a previous patient visit; and populating at least one of a qualitative risk stratification table and a quantitative risk stratification table based on the calculated rate of change for each of the PPD, OIB and DA values between the current patient visit and a previous patient visit; whereby, one or both of the qualitative risk stratification table and quantitative risk stratification table can be used by the medical service provider to accurately and objectively predict the patient's future risk of periodontal disease.

62. The method according to embodiment 61, wherein the step of determining a quantity of total MMP-8 along with a quantity of at least one of active MMP-8 and latent MMP8, further comprises the steps of: determining a quantity of an at least one ubiquitous protein present in the collected saliva; and calculating a specific activity of at least one of the total MMP-8, active MMP-8 and latent MMP-8 using the formula

$$Specific Activity = \frac{MMP - 8}{ubiquitous protein} .$$

63. The method according to embodiments 61-62, wherein the step of determining a quantity of total MMP-8 along with a quantity of at least one of active MMP-8 and latent MMP8, further comprises the step of analyzing the collected saliva using an enzyme-linked immunosorbent assay test.

64. The method according to embodiments 61-63, wherein the step of determining a quantity of total MMP-8 along with a quantity of at least one of active MMP-8 and latent MMP8, further comprises the step of analyzing the collected saliva using a lateral flow assay test.

65. The method according to embodiments 61-64, wherein the step of collecting a volume of the patient's saliva further comprises the step of collecting a volume of the patient's saliva prior to performing any treatments or clinical measurements on the patient.

66. The method according to embodiments 61-65, wherein the step of calculating a disease activity further comprises the steps of: calculating a disease activity for sites where bleeding on probing is detected; and calculating a disease activity for sites where bleeding on probing is not detected.

67. The method according to embodiments 61-66, wherein the step of calculating a rate of change for each of the PPD, OIB and DA values between the current patient visit and a previous patient visit further comprises the steps of: calculating a rate of change for each of the PPD, OIB and DA values for sites where bleeding on probing is detected; and calculating a rate of change for each of the PPD, OIB and DA values for sites where bleeding on probing is not detected.

68. The method according to embodiments 61-67, further comprising the step of calculating an enzyme ratio, representing relative levels of active MMP-8 compared to latent MMP-8 in the collected saliva, using the formula

$${Ratio}_{a / l} = ( \frac{aMMP - 8}{lMMP - 8} )$$

where aMMP-8 is the amount of active MMP-8 concentration in the collected saliva, and lMMP-8 is the amount of latent MMP-8 concentration in the collected saliva.

69. The method according to embodiments 61-68, wherein the step of calculating an enzyme ratio further comprises the steps of: calculating an enzyme ratio for sites where bleeding on probing is detected; and calculating an enzyme ratio for sites where bleeding on probing is not detected.

70. The method according to embodiments 61-69, further comprising the step of calculating a rate of change for the enzyme ratio values between the current patient visit and a previous patient visit.

71. The method according to embodiments 61-70, wherein the step of calculating a rate of change for the enzyme ratio values between the current patient visit and a previous patient visit further comprises the steps of: calculating a rate of change for the enzyme ratio values for sites where bleeding on probing is detected; and calculating a rate of change for the enzyme ratio values for sites where bleeding on probing is not detected.

72. The method according to embodiments 61-71, further comprising the step of calculating a fold change disease activity (“FCDA”) representing a ratio in a magnitude of at least one of the OIB, PPD and DA value between the current patient visit and a previous patient visit, with said ratio divided by an amount of elapsed time between the current patient visit and the previous patient visit.

73. The method according to embodiments 61-72, wherein the step of populating at least one of a qualitative risk stratification table and a quantitative risk stratification table further comprises the step of populating a qualitative risk stratification table containing indicators denoting respective increases and decreases in the calculated rate of change for each of the PPD, OIB and DA values between the current patient visit and a previous patient visit.

74. The method according to embodiments 61-73, wherein the step of populating at least one of a qualitative risk stratification table and a quantitative risk stratification table further comprises the step of populating a quantitative risk stratification table containing the calculated rate of change for each of the PPD, OIB and DA values between the current patient visit and a previous patient visit.

75. The method according to embodiments 61-74, further comprising the step of populating a quantitative risk score table containing a risk score for each rate of change value in the quantitative risk stratification table.

76. The method according to embodiments 61-75, further comprising the step of calculating the risk score for each rate of change value in the quantitative risk stratification table by, for each of rate of change value in the quantitative risk stratification table: determining a minimum rate of change value for the corresponding one of the PPD, OIB and DA values based on all such rate of change values for all patients; determining a maximum rate of change value for the corresponding one of the PPD, OIB and DA values based on all such rate of change values for all patients; and calculating the risk score using the formula

$$Risk Score ( x ) = \frac{x - minimum value}{maximum value - minimum value} * 100$$

where x is the rate of change value in the quantitative risk stratification table for the patient, minimum value is the lowest rate of change value from all patients, and maximum value is the highest rate of change value from all patients.

77. The method according to embodiments 61-76, further comprising the step of, for each of rate of change value in the quantitative risk stratification table, calculating a distribution difference for the corresponding one of the PPD, OIB and DA values by subtracting the corresponding minimum rate of change value from the corresponding maximum rate of change value, and dividing the difference by a desired interval quantity.

78. The method according to embodiments 61-77, further comprising the steps of: categorizing the patient into a first category upon determining that the PPD value has decreased, the OIB value has decreased, and the DA value has decreased between the current patient visit and a previous patient visit, said first category indicative of the patient being at a relatively lower risk of experiencing a future adverse outcome; categorizing the patient into a second category upon determining that the PPD value has decreased, the OIB value has decreased, and the DA value has increased between the current patient visit and a previous patient visit, said second category indicative of the patient being at a relatively higher risk of experiencing a future adverse outcome; categorizing the patient into a third category upon determining that the PPD value has decreased, the OIB value has increased, and the DA value has increased between the current patient visit and a previous patient visit, said third category indicative of the patient being at a high risk of experiencing a future adverse outcome; categorizing the patient into a fourth category upon determining that the PPD value has decreased, the OIB value has increased, and the DA value has decreased between the current patient visit and a previous patient visit, said fourth category indicative of the patient being at a relatively lower risk of experiencing a near-term future adverse outcome; categorizing the patient into a fifth category upon determining that the PPD value has increased, the OIB value has increased, and the DA value has increased between the current patient visit and a previous patient visit, said fifth category indicative of the patient being at high risk of experiencing a future adverse outcome; categorizing the patient into a sixth category upon determining that the PPD value has increased, the OIB value has increased, and the DA value has decreased between the current patient visit and a previous patient visit, said sixth category indicative of the patient being at a relatively lower risk of experiencing a near-term future adverse outcome; categorizing the patient into a seventh category upon determining that the PPD value has increased, the OIB value has decreased, and the DA value has decreased between the current patient visit and a previous patient visit, said seventh category indicative of the patient potentially being at a relatively lower risk of experiencing a future adverse outcome; and categorizing the patient into an eighth category upon determining that the PPD value has increased, the OIB value has decreased, and the DA value has increased between the current patient visit and a previous patient visit, said eighth category indicative of the patient being at a moderate risk of experiencing a near-term future adverse outcome.

79. The method according to embodiments 61-78, further comprising the step of populating an MMP-8 distribution amount table containing data from each of the clinical distribution table, total MMP-8 level table and MMP-8 distribution percentage table, the MMP-8 distribution amount table containing data for both a whole mouth level of data and a site-specific level of data.

80. The method according to embodiments 61-79, wherein the step of populating an MMP-8 distribution percentage table further comprises the step of calculating the distribution percentage of active MMP-8 and the distribution percentage of latent MMP-8 for each site using the normal regression curve formula

$$f ( x ) = \frac{{L}_{Max}}{( 1 + {e}^{- k ( x - {x}_{0} )} )}$$

where e is the natural logarithm base, x₀ is the x-value of the curve's midpoint, L_{Max} is the curve's maximum value, and k is the slope of the curve.

In closing, regarding the exemplary embodiments of the present invention as shown and described herein, it will be appreciated that a saliva-based diagnostic screening system and associated methods are disclosed for performing saliva-based diagnostic screenings. Because the principles of the invention may be practiced in a number of configurations beyond those shown and described, it is to be understood that the invention is not in any way limited by the exemplary embodiments but is generally directed to a saliva-based diagnostic screening system and is able to take numerous forms to do so without departing from the spirit and scope of the invention. It will also be appreciated by those skilled in the art that the present invention is not limited to the particular geometries and materials of construction disclosed, but may instead entail other functionally comparable structures or materials, now known or later developed, without departing from the spirit and scope of the invention.

Certain embodiments of the present invention are described herein, including the best mode known to the inventor(s) for carrying out the invention. Of course, variations on these described embodiments will become apparent to those of ordinary skill in the art upon reading the foregoing description. The inventor(s) expect skilled artisans to employ such variations as appropriate, and the inventor(s) intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the terms “about” and “approximately.” As used herein, the terms “about” and “approximately” mean that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein. Similarly, as used herein, unless indicated to the contrary, the term “substantially” is a term of degree intended to indicate an approximation of the characteristic, item, quantity, parameter, property, or term so qualified, encompassing a range that can be understood and construed by those of ordinary skill in the art.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (along with equivalent open-ended transitional phrases thereof such as “including,” “containing” and “having”) encompasses all the expressly recited elements, limitations, steps and/or features alone or in combination with un-recited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amendment for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim, whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (along with equivalent open-ended transitional phrases thereof) includes within its meaning, as a limiting case, claimed subject matter specified by the closed-ended transitional phrases “consisting of” or “consisting essentially of.” As such, embodiments described herein or so claimed with the phrase “comprising” are expressly or inherently unambiguously described, enabled and supported herein for the phrases “consisting essentially of” and “consisting of.”

Any claims intended to be treated under 35 U.S.C. § 112(f) will begin with the words “means for,” but use of the term “for” in any other context is not intended to invoke treatment under 35 U.S.C. § 112(f). Accordingly, Applicant reserves the right to pursue additional claims after filing this application, in either this application or in a continuing application.

It should be understood that the logic code, programs, modules, processes, methods, and the order in which the respective elements of each method are performed are purely exemplary. Depending on the implementation, they may be performed in any order or in parallel, unless indicated otherwise in the present disclosure. Further, the logic code is not related, or limited to any particular programming language, and may comprise one or more modules that execute on one or more processors in a distributed, non-distributed, or multiprocessing environment. Additionally, the various illustrative logical blocks, modules, methods, and algorithm processes and sequences described in connection with the embodiments disclosed herein can be implemented as electronic hardware, computer software, or combinations of both. To clearly illustrate this interchangeability of hardware and software, various illustrative components, blocks, modules, and process actions have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of this document.

The phrase “non-transitory,” in addition to having its ordinary meaning, as used in this document means “enduring or long-lived”. The phrase “non-transitory computer readable medium,” in addition to having its ordinary meaning, includes any and all computer readable mediums, with the sole exception of a transitory, propagating signal. This includes, by way of example and not limitation, non-transitory computer-readable mediums such as register memory, processor cache and random-access memory (“RAM”).

The methods as described above may be used in the fabrication of integrated circuit chips. The resulting integrated circuit chips can be distributed by the fabricator in raw wafer form (that is, as a single wafer that has multiple unpackaged chips), as a bare die, or in a packaged form. In the latter case, the chip is mounted in a single chip package (such as a plastic carrier, with leads that are affixed to a motherboard or other higher level carrier) or in a multi-chip package (such as a ceramic carrier that has either or both surface interconnections or buried interconnections). In any case, the chip is then integrated with other chips, discrete circuit elements, and/or other signal processing devices as part of either (a) an intermediate product, such as a motherboard, or (b) an end product. The end product can be any product that includes integrated circuit chips, ranging from toys and other low-end applications to advanced computer products having a display, a keyboard or other input device, and a central processor.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

While aspects of the invention have been described with reference to at least one exemplary embodiment, it is to be clearly understood by those skilled in the art that the invention is not limited thereto. Rather, the scope of the invention is to be interpreted only in conjunction with the appended claims and it is made clear, here, that the inventor(s) believe that the claimed subject matter is the invention.

What is claimed is:

1. A method for screening a volume of saliva of a patient for a presence or absence of active periodontal disease, the method comprising the steps of:
implementing a user application residing in memory on an at least one computing device, the at least one computing device configured for receiving and processing select data, obtained by an at least one saliva screening device, based on the patient's saliva; and
for each of an at least one patient visit to a medical service provider of the patient:
collecting a volume of the patient's saliva;
the user application determining a quantity of total MMP-8 along with a quantity of at least one of active MMP-8 and latent MMP-8 present in the collected saliva;
the user application populating a clinical measurement table containing a plurality of probing pocket depth (“PPD”) values and corresponding bleeding on probing (“BOP”) values for an at least one site within a mouth of the patient as measured by a medical service provider for the patient;
the user application populating a clinical distribution table containing a distribution of a quantity of BOP sites relative to each PPD value in the clinical measurement table;
the user application calculating a weighted average PPD for each site where bleeding on probing is detected using the formula
$$f ( x ) = {\sum }_{x = 1}^{14} ( PPD ( x ) \times BOP Sites ( x ) ) / {\sum }_{x = 1}^{14} BOP Sites ( x )$$
where x is the PPD value corresponding to each site;
the user application calculating a weighted average PPD for each site where bleeding on probing is not detected using the formula
$$f ( x ) = {\sum }_{x = 1}^{14} ( PPD ( x ) \times noBOP Sites ( x ) ) / {\sum }_{x = 1}^{14} noBOP Sites ( x )$$
where x is the PPD value corresponding to each site;
the user application calculating a distribution of total MMP-8 quantities for each site where bleeding on probing is detected using the formula
$$f ( x ) = ( \frac{BOP Sites ( x )}{total sites} ) \times ( tMMP - 8 concentration )$$
where x is the PPD value corresponding to each site;
the user application calculating a distribution of total MMP-8 quantities for each site where bleeding on probing is not detected using the formula
$$f ( x ) = ( \frac{noBOP Sites ( x )}{total sites} ) \times ( tMMP - 8 concentration )$$
where x is the PPD value corresponding to each site;
the user application populating a total MMP-8 level table containing the calculated weighted average PPD and distribution of total MMP-8 quantities for each site;
the user application populating an MMP-8 distribution percentage table containing a distribution percentage of active MMP-8 and a distribution percentage of latent MMP-8 for each site based;
the user application calculating an oral inflammatory burden (“OIB”) based on the level of total MMP-8 in the collected saliva;
the user application calculating a disease activity (“DA”), representing a relative level of active MMP-8 compared to the level of total MMP-8 in the collected saliva, using the formula
$$DA = ( \frac{aMMP - 8}{tMMP - 8} )$$
where aMMP-8 is the amount of active MMP-8 concentration in the collected saliva and tMMP-8 is the amount of total MMP-8 concentration in the collected saliva;
the user application calculating a rate of change for each of the PPD, OIB and DA values between the current patient visit and a previous patient visit; and
the user application populating at least one of a qualitative risk stratification table and a quantitative risk stratification table based on the calculated rate of change for each of the PPD, OIB and DA values between the current patient visit and a previous patient visit;
whereby, one or both of the qualitative risk stratification table and quantitative risk stratification table can be used by the medical service provider to accurately and objectively predict the patient's future risk of periodontal disease.

2. The method of claim 1, wherein the step of the user application determining a quantity of total MMP-8 along with a quantity of at least one of active MMP-8 and latent MMP8, further comprises the steps of:
the user application determining a quantity of an at least one ubiquitous protein present in the collected saliva; and
the user application calculating a specific activity of at least one of the total MMP-8, active MMP-8 and latent MMP-8 using the formula
$$Specific = \frac{MMP - 8}{ubiquitous protein} .$$

3. The method of claim 1, further comprising the step of the user application calculating an enzyme ratio, representing relative levels of active MMP-8 compared to latent MMP-8 in the collected saliva, using the formula
$${Ratio}_{a / l} = ( \frac{aMMP - 8}{lMMP - 8} )$$
where aMMP-8 is the amount of active MMP-8 concentration in the collected saliva, and lMMP-8 is the amount of latent MMP-8 concentration in the collected saliva.

4. The method of claim 3, further comprising the step of the user application calculating a rate of change for the enzyme ratio values between the current patient visit and a previous patient visit.

5. The method of claim 1, wherein the step of the user application populating at least one of a qualitative risk stratification table and a quantitative risk stratification table further comprises the step of the user application populating a qualitative risk stratification table containing indicators denoting respective increases and decreases in the calculated rate of change for each of the PPD, OIB and DA values between the current patient visit and a previous patient visit.

6. The method of claim 1, wherein the step of the user application populating at least one of a qualitative risk stratification table and a quantitative risk stratification table further comprises the step of the user application populating a quantitative risk stratification table containing the calculated rate of change for each of the PPD, OIB and DA values between the current patient visit and a previous patient visit.

7. The method of claim 6, further comprising the step of the user application populating a quantitative risk score table containing a risk score for each rate of change value in the quantitative risk stratification table.

8. The method of claim 7, further comprising the step of the user application calculating the risk score for each rate of change value in the quantitative risk stratification table by, for each of rate of change value in the quantitative risk stratification table:
the user application determining a minimum rate of change value for the corresponding one of the PPD, OIB and DA values based on all such rate of change values for all patients;
the user application determining a maximum rate of change value for the corresponding one of the PPD, OIB and DA values based on all such rate of change values for all patients; and
the user application calculating the risk score using the formula
$$Risk Score ( x ) = \frac{x - minimum value}{maximum value - minimum value} * 100$$
where x is the rate of change value in the quantitative risk stratification table for the patient, minimum value is the lowest rate of change value from all patients, and maximum value is the highest rate of change value from all patients.

9. The method of claim 8, further comprising the step of the user application, for each of rate of change value in the quantitative risk stratification table, calculating a distribution difference for the corresponding one of the PPD, OIB and DA values by subtracting the corresponding minimum rate of change value from the corresponding maximum rate of change value, and dividing the difference by a desired interval quantity.

10. The method of claim 1, further comprising the steps of:
the user application categorizing the patient into a first category upon determining that the PPD value has decreased, the OIB value has decreased, and the DA value has decreased between the current patient visit and a previous patient visit, said first category indicative of the patient being at a relatively lower risk of experiencing a future adverse outcome;
the user application categorizing the patient into a second category upon determining that the PPD value has decreased, the OIB value has decreased, and the DA value has increased between the current patient visit and a previous patient visit, said second category indicative of the patient being at a relatively higher risk of experiencing a future adverse outcome;
the user application categorizing the patient into a third category upon determining that the PPD value has decreased, the OIB value has increased, and the DA value has increased between the current patient visit and a previous patient visit, said third category indicative of the patient being at a high risk of experiencing a future adverse outcome;
the user application categorizing the patient into a fourth category upon determining that the PPD value has decreased, the OIB value has increased, and the DA value has decreased between the current patient visit and a previous patient visit, said fourth category indicative of the patient being at a relatively lower risk of experiencing a near-term future adverse outcome;
the user application categorizing the patient into a fifth category upon determining that the PPD value has increased, the OIB value has increased, and the DA value has increased between the current patient visit and a previous patient visit, said fifth category indicative of the patient being at high risk of experiencing a future adverse outcome;
the user application categorizing the patient into a sixth category upon determining that the PPD value has increased, the OIB value has increased, and the DA value has decreased between the current patient visit and a previous patient visit, said sixth category indicative of the patient being at a relatively lower risk of experiencing a near-term future adverse outcome;
the user application categorizing the patient into a seventh category upon determining that the PPD value has increased, the OIB value has decreased, and the DA value has decreased between the current patient visit and a previous patient visit, said seventh category indicative of the patient potentially being at a relatively lower risk of experiencing a future adverse outcome; and
the user application categorizing the patient into an eighth category upon determining that the PPD value has increased, the OIB value has decreased, and the DA value has increased between the current patient visit and a previous patient visit, said eighth category indicative of the patient being at a moderate risk of experiencing a near-term future adverse outcome.

11. A saliva-based diagnostic screening system for screening a volume of saliva of a patient for a presence or absence of active periodontal disease, the system comprising:
a user application residing in memory on an at least one computing device, the at least one computing device configured for receiving and processing select data, obtained by an at least one saliva screening device, based on the patient's saliva;
wherein, for each of an at least one patient visit to a medical service provider of the patient, the user application is configured for:
determining a quantity of total MMP-8 along with a quantity of at least one of active MMP-8 and latent MMP-8 present in a volume of saliva collected from the patient;
populating a clinical measurement table containing a plurality of probing pocket depth (“PPD”) values and corresponding bleeding on probing (“BOP”) values for an at least one site within a mouth of the patient as measured by a medical service provider for the patient;
populating a clinical distribution table containing a distribution of a quantity of BOP sites relative to each PPD value in the clinical measurement table;
calculating a weighted average PPD for each site where bleeding on probing is detected using the formula
$$f ( x ) = {\sum }_{x = 1}^{14} ( PPD ( x ) \times BOP Sites ( x ) ) / {\sum }_{x = 1}^{14} BOP Sites ( x )$$
where x is the PPD value corresponding to each site;
calculating a weighted average PPD for each site where bleeding on probing is not detected using the formula
$$f ( x ) = {\sum }_{x = 1}^{14} ( PPD ( x ) \times noBOP Sites ( x ) ) / {\sum }_{n = 1}^{14} noBOP Sites ( x )$$
where x is the PPD value corresponding to each site;
calculating a distribution of total MMP-8 quantities for each site where bleeding on probing is detected using the formula
$$f ( x ) = ( \frac{BOP Sites ( x )}{total sites} ) \times ( tMMP - 8 concentration )$$
where x is the PPD value corresponding to each site;
calculating a distribution of total MMP-8 quantities for each site where bleeding on probing is not detected using the formula
$$f ( x ) = ( \frac{noBOP Sites ( x )}{total sites} ) \times ( tMMP - 8 concentration )$$
where x is the PPD value corresponding to each site;
populating a total MMP-8 level table containing the calculated weighted average PPD and distribution of total MMP-8 quantities for each site;
populating an MMP-8 distribution percentage table containing a distribution percentage of active MMP-8 and a distribution percentage of latent MMP-8 for each site;
calculating an oral inflammatory burden (“OIB”) based on the level of total MMP-8 in the collected saliva;
calculating a disease activity (“DA”), representing a relative level of active MMP-8 compared to the level of total MMP-8 in the collected saliva, using the formula
$$DA = ( \frac{aMMP - 8}{tMMP - 8} )$$
where aMMP-8 is the amount of active MMP-8 concentration in the collected saliva and tMMP-8 is the amount of total MMP-8 concentration in the collected saliva;
calculating a rate of change for each of the PPD, OIB and DA values between the current patient visit and a previous patient visit; and
populating at least one of a qualitative risk stratification table and a quantitative risk stratification table based on the calculated rate of change for each of the PPD, OIB and DA values between the current patient visit and a previous patient visit;
whereby, one or both of the qualitative risk stratification table and quantitative risk stratification table can be used by the medical service provider to accurately and objectively predict the patient's future risk of periodontal disease.

12. A method for screening a volume of saliva of a patient for a presence or absence of active periodontal disease, the method comprising the steps of, for each of an at least one patient visit to a medical service provider of the patient:
collecting a volume of the patient's saliva;
determining a quantity of total MMP-8 along with a quantity of at least one of active MMP-8 and latent MMP-8 present in the collected saliva;
populating a clinical measurement table containing a plurality of probing pocket depth (“PPD”) values and corresponding bleeding on probing (“BOP”) values for an at least one site within a mouth of the patient as measured by a medical service provider for the patient;
populating a clinical distribution table containing a distribution of a quantity of BOP sites relative to each PPD value in the clinical measurement table;
calculating a weighted average PPD for each site where bleeding on probing is detected using the formula
$$f ( x ) = {\sum }_{x = 1}^{14} ( PPD ( x ) \times BOP Sites ( x ) ) / {\sum }_{x = 1}^{14} BOP Sites ( x )$$
where x is the PPD value corresponding to each site;
calculating a weighted average PPD for each site where bleeding on probing is not detected using the formula
$$f ( x ) = {\sum }_{x = 1}^{14} ( PPD ( x ) \times noBOP Sites ( x ) ) / {\sum }_{n = 1}^{14} noBOP Sites ( x )$$
where x is the PPD value corresponding to each site;
calculating a distribution of total MMP-8 quantities for each site where bleeding on probing is detected using the formula
$$f ( x ) = ( \frac{BOP Sites ( x )}{total sites} ) \times ( tMMP - 8 concentration )$$
where x is the PPD value corresponding to each site;
calculating a distribution of total MMP-8 quantities for each site where bleeding on probing is not detected using the formula
$$f ( x ) = ( \frac{noBOP Sites ( x )}{total sites} ) \times ( tMMP - 8 concentration )$$
where x is the PPD value corresponding to each site;
populating a total MMP-8 level table containing the calculated weighted average PPD and distribution of total MMP-8 quantities for each site;
populating an MMP-8 distribution percentage table containing a distribution percentage of active MMP-8 and a distribution percentage of latent MMP-8 for each site;
calculating an oral inflammatory burden (“OIB”) based on the level of total MMP-8 in the collected saliva;
calculating a disease activity (“DA”), representing a relative level of active MMP-8 compared to the level of total MMP-8 in the collected saliva, using the formula
$$DA = ( \frac{aMMP - 8}{tMMP - 8} )$$
where aMMP-8 is the amount of active MMP-8 concentration in the collected saliva and tMMP-8 is the amount of total MMP-8 concentration in the collected saliva;
calculating a rate of change for each of the PPD, OIB and DA values between the current patient visit and a previous patient visit; and
populating at least one of a qualitative risk stratification table and a quantitative risk stratification table based on the calculated rate of change for each of the PPD, OIB and DA values between the current patient visit and a previous patient visit;
whereby, one or both of the qualitative risk stratification table and quantitative risk stratification table can be used by the medical service provider to accurately and objectively predict the patient's future risk of periodontal disease.

13. The method of claim 12, further comprising the step of calculating an enzyme ratio, representing relative levels of active MMP-8 compared to latent MMP-8 in the collected saliva, using the formula
$${Ratio}_{a / l} = ( \frac{aMMP - 8}{lMMP - 8} )$$
where aMMP-8 is the amount of active MMP-8 concentration in the collected saliva, and lMMP-8 is the amount of latent MMP-8 concentration in the collected saliva.

14. The method of claim 13, further comprising the step of calculating a rate of change for the enzyme ratio values between the current patient visit and a previous patient visit.

15. The method of claim 1, wherein the step of populating at least one of a qualitative risk stratification table and a quantitative risk stratification table further comprises the step of populating a qualitative risk stratification table containing indicators denoting respective increases and decreases in the calculated rate of change for each of the PPD, OIB and DA values between the current patient visit and a previous patient visit.

16. The method of claim 1, wherein the step of populating at least one of a qualitative risk stratification table and a quantitative risk stratification table further comprises the step of populating a quantitative risk stratification table containing the calculated rate of change for each of the PPD, OIB and DA values between the current patient visit and a previous patient visit.

17. The method of claim 16, further comprising the step of populating a quantitative risk score table containing a risk score for each rate of change value in the quantitative risk stratification table.

18. The method of claim 17, further comprising the step of calculating the risk score for each rate of change value in the quantitative risk stratification table by, for each of rate of change value in the quantitative risk stratification table:
determining a minimum rate of change value for the corresponding one of the PPD, OIB and DA values based on all such rate of change values for all patients;
determining a maximum rate of change value for the corresponding one of the PPD, OIB and DA values based on all such rate of change values for all patients; and
calculating the risk score using the formula
$$Risk Score ( x ) = \frac{x - minimum value}{maximum value - minimum value} * 100$$
where x is the rate of change value in the quantitative risk stratification table for the patient, minimum value is the lowest rate of change value from all patients, and maximum value is the highest rate of change value from all patients.

19. The method of claim 18, further comprising the step of, for each of rate of change value in the quantitative risk stratification table, calculating a distribution difference for the corresponding one of the PPD, OIB and DA values by subtracting the corresponding minimum rate of change value from the corresponding maximum rate of change value, and dividing the difference by a desired interval quantity.

20. The method of claim 1, further comprising the steps of:
categorizing the patient into a first category upon determining that the PPD value has decreased, the OIB value has decreased, and the DA value has decreased between the current patient visit and a previous patient visit, said first category indicative of the patient being at a relatively lower risk of experiencing a future adverse outcome;
categorizing the patient into a second category upon determining that the PPD value has decreased, the OIB value has decreased, and the DA value has increased between the current patient visit and a previous patient visit, said second category indicative of the patient being at a relatively higher risk of experiencing a future adverse outcome;
categorizing the patient into a third category upon determining that the PPD value has decreased, the OIB value has increased, and the DA value has increased between the current patient visit and a previous patient visit, said third category indicative of the patient being at a high risk of experiencing a future adverse outcome;
categorizing the patient into a fourth category upon determining that the PPD value has decreased, the OIB value has increased, and the DA value has decreased between the current patient visit and a previous patient visit, said fourth category indicative of the patient being at a relatively lower risk of experiencing a near-term future adverse outcome;
categorizing the patient into a fifth category upon determining that the PPD value has increased, the OIB value has increased, and the DA value has increased between the current patient visit and a previous patient visit, said fifth category indicative of the patient being at high risk of experiencing a future adverse outcome;
categorizing the patient into a sixth category upon determining that the PPD value has increased, the OIB value has increased, and the DA value has decreased between the current patient visit and a previous patient visit, said sixth category indicative of the patient being at a relatively lower risk of experiencing a near-term future adverse outcome;
categorizing the patient into a seventh category upon determining that the PPD value has increased, the OIB value has decreased, and the DA value has decreased between the current patient visit and a previous patient visit, said seventh category indicative of the patient potentially being at a relatively lower risk of experiencing a future adverse outcome; and
categorizing the patient into an eighth category upon determining that the PPD value has increased, the OIB value has decreased, and the DA value has increased between the current patient visit and a previous patient visit, said eighth category indicative of the patient being at a moderate risk of experiencing a near-term future adverse outcome.
